(19) United States
(12) Patent Application Publication
(10) Pub. No.: US 2006/0172363 A1
(43) Pub. Date: Aug. 3, 2006

(54) Peptide complexes containing phospholipase d

(75) Inventors: Sung Ho Ryu, Pohang-city (KR); Pann Ghill Suh, Pohang-city (KR); Jong Hyun Kim, Pohang-city (JP); Il Ho Jang, Pohang-city (KR); Hye Young Lee, Pohang-city (KR); Young Chan Chae, Pohang-city (KR); Sang Hoon Ha, Pohang-city (KR); Jong Bae Park, Pohang-city (KR); Jung Hwan Kim, Pohang-city (KR); Sukmook Lee, Seoul (KR); Jun Sung Lee, Pohang-city (KR); Chang Sup Lee, Pohang-city (KR); Hyun Wook Kim, Pohang-city (KR); Il Shin Kim, Pohang-city (KR); Hyeona Jeon, Pohang-city (KR)

Correspondence Address: ROTHWELL, FIGG, ERNST & MANBECK, P.C., 1425 K STREET, N.W., SUITE 800, WASHINGTON, DC, 20005, US

(73) Assignee: POSTECH FOUNDATION, Pohang-city (KR)

(21) Appl. No.: 10/528,172

(22) PCT Filed: Sep. 18, 2003
(86) PCT No.: PCT/KR03/01903

§ 371 Date: Mar. 17, 2005

Related U.S. Application Data
(60) Provisional application No. 60/411,600, filed on Sep. 18, 2002.
(60) Provisional application No. 60/416,552, filed on Oct. 8, 2002.

(51) Int. Cl. C12Q 1/34; G06F 19/00; C12N 9/20

(52) U.S. Cl. 435/18; 435/198; 702/19

(57) ABSTRACT

The present invention provides peptide complexes comprising phospholipase D and one or morephospholipase D-interacting peptides. The peptide complexes are useful in screening assays foridentifying compounds effective in modulating the peptide complexes and in treating and/or preventing diseases associated with phospholipase D and it's interacting partners. In addition, methods for screening modulators of the peptide complexes or interacting members thereof are provided.

### CROSS REFERENCE TO RELATED APPLICATIONS

This application is a 35 U.S.C. § 371 National Phase Entry Application from PCT/KR03/001903, filed Sep. 18, 2003, designating the U.S. which claims benefit of U.S. Provisional Application 60/411,600 filed on Sep. 18, 2002 and U.S. Provisional Application 60/416,552 filed on Oct. 8, 2002.

### BACKGROUND OF THE INVENTION

1. Field of the Invention

The present invention relates to an isolated peptide complex, more specifically, to an isolated peptide complex comprising phospholipase D, and a screening method for modulators thereof.

2. Background Art Description of the Related Art

Mammalian phospholipase D (PLD) is an enzyme that hydrolyzes phosphatidyl choline (PC) into phosphatidic acid (PA) and choline in response to a variety of signals including hormones, neurotransmitters, and growth factors. PA is known as intracellular lipid second messengers, which are involved in multiple physiological events such as promotion of mitogenesis, stimulation of respiratory bursts, secretory processes, and actin cytoskeletal reorganization in many cells (Lee, S., et al., J. Biol. Chem., 277:6542-6549 (2002)). PA can be also converted into various other signaling molecules such as diacylglycerol (DAG), lysophosphatidic acid (LPA), and arachidonate.

There are two mammalian PLD isoforms, PLD1 and PLD2. PLD2 is ˜50% identical to PLD1. Unless otherwise specified, the term “PLD” used herein encompasses both PLD1 and PLD2. The essential feature that defines mammalian PLD is the presence of two catalytic domains. In addition, both mammalian PLDs contain a putative pleckstrin homology (PH) domain, a phox homology (PX) domain, and other conserved regions of unknown function. PLD1 contains a “loop” sequence between two catalytic domains, which is not found in PLD2 (Rizzo, M. A., et al., Pharmacol. Therapeutics, 94:35-50 (2002)). PLD activity in mammalian cells is regulated by various factors including proteins and lipids, for example, protein kinase C (PKC), small GTPases, and phosphatidylinositol 4,5-bisphosphate (PIP2) (Melendez, A. et al., Immunology, 14:49-55 (2002)). This indicates that PLD regulation is a complicated and tightly regulated process. The ubiquitous nature of agonist-dependent PLD activation suggests that PLD is involved in the regulation of important cellular processes and it may function as a hub molecule in cellular signaling (Exton J H, Biochim. Biophys. Acta, 1439, 121-133 (1997)).

Further, PLD is involved in budding, intracellular vesicle trafficking and vacuolar molecule sorting, formation of multivesicular bodies, endocytosis, tumorigenesis, cell transformation, and proliferation (Foster D A, Mol Cell Biol 21:595-602 (2001), Morris A J, Biochim Biophys Acta, 1439:175-86 (1999)) and it is thus associated with diseases and disorders such as neurodegenerative diseases, autoimmune diseases, cancer, and diabetes (Min D S, J Biol Chem. 277:12334-42 (2002), Farese R V, Am J Physiol Endocrinol Metab., 283:E1-11 (2002)).

Therefore, it is highly valuable to find a novel PLD binding partner and/or a peptide complex of PLD and the PLD binding partner. That is, it can be presumed that a peptide complex of PLD and a PLD binding partner mediates the functions of PLD and the binding partners in the biological processes or disease pathways. Therefore, a peptide complex comprising PLD and a PLD binding partner will be useful for modulating biological processes or treating diseases by PLD (Smith S O, J Biol Chem., 278: 21459-66. (2003)). Further, a peptide complex comprising PLD and a PLD binding partner will be useful for screening a modulator of an interaction between PLD and a PLD binding partner, which may be also used to modulate biological processes or to treat diseases with PLD. As used herein, modulating an interaction between PLD and a PLD binding partner means altering (enhancing or reducing) the activities of PLD and/or a PLD binding partner, e.g., increasing the concentrations of PLD and/or a PLD binding partner, enhancing or reducing their biological activities, increasing or decreasing their stability, altering their affinity or specificity to certain other biological molecules, etc.

For example, a modulator of the peptide complex, including agonist and antagonist, would be useful in treating diseases and disorders such as neurodegenerative diseases, autoimmune diseases, cancer and diabetes.

Therefore, undoubtedly, there is a continued need to develop a peptide complex comprising PLD and a PLD binding partner.

### SUMMARY OF THE INVENTION

The present invention provides novel PLD binding partners, with which PLD forms a peptide complex useful for modulating biological processes or treating diseases by PLD and/or the PLD binding partners and a method for screening modulators of the peptide complex.

In one aspect of the present invention, there is provided an isolated peptide complex comprising a first peptide selected from the group consisting of (a1) phospholipase D (PLD), (a2) a PLD variant, (a3) a PLD fragment, and (a4) a fusion peptide containing (a1), (a2), or (a3); and a second peptide selected from the group consisting of (b1) actin, aldolase, collapsin response mediator molecule-2 (CRMP-2), phospholipase Cγ-1 (PLC-γ1), glyceraldehyde-3-phosphate dehydrolase (GAPDH), Akt1, glucose transporter 4 (GLUT4), mammalian target of rapamycin (mTOR), heat shock protein 70 (hsp7o), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), or dopamine transporter (DAT), (b2) a variant of (b1), (b3) a fragment of (b1), and (b4) a fusion peptide containing (b1), (b2), or (b3). The first peptide may be linked to the second peptide by a covalent bond.

In another aspect of the present invention, there is provided a screening method for modulators of the peptide complex, which comprises providing the isolated peptide complex; contacting the isolated peptide complex with a test compound; and detecting an interaction between the test compound and the isolated peptide complex and/or an interaction change between the first peptide and the second peptide.

In still another aspect of the present invention, there is provided a screening method for modulators of an interaction between the first peptide and the second peptide, which comprises contacting the first peptide with the second peptide in the presence of a test compound; and detecting an interaction between the first peptide and the second peptide.

### BRIEF DESCRIPTION OF THE DRAWINGS

The above and other features and advantages of the present invention will become more apparent by describing in detail exemplary embodiments thereof with reference to the attached drawings in which:

FIG. 1 shows detection of a 43-kDa protein as a PLD2-binding protein;

FIG. 2 shows interaction between actin and PLD2;

FIG. 3A shows the isolation of PLD2-binding protein (p40) by the above blot overlay assay;

FIG. 3B shows the matrix-assisted laser desorption ionization mass spectrum of the digested peptides of p40;

FIG. 4 shows direct interaction between aldolase and PLD2;

FIG. 5 shows detection of a p62 as a PLD2-binding protein;

FIG. 6 shows direct interaction between CRMP-2 and PLD2-PX;

FIG. 7 shows direct interaction between PLC-γ1 and PLD2;

FIG. 8 shows detection of a p35 as a PLD2-binding protein;

FIG. 9 shows direct interaction between Akt and PLD2;

FIG. 10 shows association with PLD1 and GLUT4;

FIG. 11 shows a direct binding of purified PLD1 to the cytoplasmic central loop of GLUT4;

FIGS. 12 and 13 show detection of HSP70 as a PLD2-binding protein;

FIG. 14 shows direct interaction between HSP70 and PLD2;

FIGS. 15A and 15B show detection of dynamin as a PLD-binding protein;

FIG. 16 shows direct interaction between dynamin and PLD2;

FIG. 17 shows detection of munc-18-1 as a PLD -binding protein;

FIG. 18 shows direct interaction between munc-18-1 and PLD2;

FIG. 19 shows detection of p55 protein as a PLD2-binding protein;

FIG. 20 shows that the 55-kDa protein is co-precipitated with PLD2 in COS-7 cells is α- and β-tubulin;

FIG. 21 shows detection of nNOS as a PLD2-binding protein;

FIG. 22 shows interaction between nNOS and PLD2;

FIG. 23 shows direct interaction between integrin beta 3, 5 cytosolic tail and PLD2;

FIG. 24 shows interaction between integrin beta 3 and PLD2;

FIG. 25 shows an interaction between mTOR and PLD2; and

FIGS. 26A, 26B, and 26C show affinity between PLD1 and phosphoinositide-3-phosphate (PIP3).

### DETAILED DESCRIPTION OF THE INVENTION

The terms “polypeptide,” “molecule,” and “peptide” are used herein interchangeably to refer to amino acid chains in which the amino acid residues are linked by peptide bonds or modified peptide bonds. The amino acid chains can be of any length greater than two amino acids. Unless otherwise specified, the terms “polypeptide,” “molecule,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent bonds to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

As used herein, the term “interacting” or “interaction” means that two molecule domains, fragments or complete molecules exhibit sufficient physical affinity to each other so as to bring the two “interacting” molecule domains, fragments or molecules physically close to each other. Such interaction can be attained by chemical bond(s) or based solely on physical affinities. Examples of physical affinities and chemical bonds include, but are not limited to, forces caused by electrical charge differences, hydrophobicity, hydrogen bonds, van der Waals force, ionic force, covalent linkages, and combination(s) thereof. The state of proximity between the interaction domains, fragments, molecules or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, molecules, or entities. Examples of interactions include specific interactions between antigen and antibody, ligand and receptor, enzyme and substrate, and the like.

As used herein, the term “peptide complex” means a composite unit that is a combination of two or more peptides formed by interaction between the peptides. Typically, but not necessarily, a “peptide complex” is formed by the binding of two or more peptides together through specific non-covalent interactions. However, covalent bonds may also be present between the interacting partners. For instance, the two interacting partners can be covalently crosslinked so that the peptide complex becomes more stable.

The term “peptide fragment” as used herein means a polypeptide that represents a portion of a peptide. When a peptide fragment exhibits interactions with another peptide or peptide fragment, the two entities are said to interact through interaction domains that are contained within the entities.

The term “isolated peptide complex” means a peptide complex present in a composition or environment that is different from that found in nature—in its native or original cellular or body environment. Preferably, an “isolated peptide complex” is separated from at least 50%, more preferably at least 75%, most preferably at least 90%, of other naturally co-existing cellular or tissue components. Thus, an “isolated peptide complex” may also be a naturally existing peptide complex in an artificial preparation or a non-native host cell. An “isolated peptide complex” may also be a “purified peptide complex”, that is, a substantially purified form in a substantially homogenous preparation substantially free of other cellular components, other polypeptides, viral materials, or culture medium, or, when the peptide components in the peptide complex are chemically synthesized, free of chemical precursors or by-products associated with the chemical synthesis. A “purified peptide complex” typically means a preparation containing preferably at least 75%, more preferably at least 85%, and most preferably at least 95%, of a particular peptide complex. A “purified peptide complex” may be obtained from natural or recombinant host cells or other body samples by standard purification techniques, or by chemical synthesis.

The term “fusion peptide” used herein to mean a non-naturally occurring peptide having a specified polypeptide molecule covalently linked to one or more polypeptide molecules that do not naturally link to the specified polypeptide. Thus, a “fusion peptide” may include two naturally occurring molecules or fragments thereof linked together by a covalent linkage. A “fusion peptide” may also be-a peptide formed—by covalently linking two artificial polypeptides together. Typically but not necessarily, the two or more polypeptides are linked or “fused” together by a peptide bond forming a single non-branched polypeptide chain.

As used herein, the term “modulator” encompasses any compounds that can cause any forms of alteration of the biological activities or functions of the peptides or peptide complexes, including, e.g., enhancing or reducing their biological activities, increasing or decreasing their stability, altering their affinity or specificity to certain other biological peptides, etc. In addition, the term “modulator” as used herein also includes any compounds that simply bind PLD, PLD-interacting peptides, and/or the peptides complexes of the present invention. For example, a modulator can be an “interaction antagonist” capable of interfering with or disrupting or dissociating peptide-peptide interaction between PLD or a homologue, fragment or derivative thereof and one or more peptides selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) or a homologue, fragment or derivative thereof. A modulator can also be an “interaction agonist” that initiates or strengthens the interaction between the peptide members of a peptide complex of the present invention, or homologues, fragments or derivatives thereof.

The present invention provides an isolated peptide complex comprising: a first peptide selected from the group consisting of phospholipase D (PLD), a PLD variant, a PLD fragment, and a fusion peptide containing PLD, a PLD variant, or a PLD fragment; and a second peptide selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) a variant thereof, a fragment thereof, and a fusion peptide thereof.

The second peptide has a specific binding affinity to PLD, thus forming a peptide complex with PLD. β-Actin, a 43-kDa protein in the rat brain, acts as a major PLD2 direct-binding protein as revealed by peptide mass fingerprinting in combination with matrix-assisted laser desorption ionization/time-of-flight mass spectrometry. The region between amino acids 613 and 723 of PLD2 is required for the direct binding of β-actin, using bacterially expressed glutathione S-transferase fusion proteins of PLD2 fragments. Purified β-actin is known to potently inhibit both phosphatidylinositol-4,5-bisphosphate- and oleate-dependent PLD2 activities in a concentration-dependent manner (IC₅₀=5 nM). It had been previously reported that α-actinin inhibited PLD2 activity in an interaction-dependent and an ADP-ribosylation factor 1 (ARF1)-reversible manner (Park, J. B., Kim, J. H., Kim, Y., Ha, S. H., Kim, J. H., Yoo, J.-S., Du, G., Frohman, M. A., Suh, P.-G., and Ryu, S. H. (2000) J. Biol. Chem. 275, 21295-21301). In vitro binding analyses showed that β-actin could displace α-actinin binding to PLD2, demonstrating independent interaction between cytoskeletal proteins and PLD2. Furthermore, ARF1 could steer PLD2 activity in a positive direction regardless of the inhibitory effect of β-actin on PLD2. We also observed that β-actin regulates PLD1 and PLD2 with similar binding and inhibitory potencies. Immunocytochemical and co-immunoprecipitation studies demonstrated in vivo interaction between the two PLD isozymes and actin in cells. Taken together, such results indicate that the regulation of PLD by cytoskeletal proteins, β-actin and α-actinin, and ARF1 may play an important role in cytoskeleton-related PLD functions.

Aldolase A also interacts with PLD. To identify the peptides that interact with PLD, a peptide overlay assay was performed with fractions obtained from the sequential column chromatographic separation of rat brain cytosol using purified PLD2 as a probe. A peptide of molecular weight 40 kDa, which was detected by anti-PLD antibody with overlaying the purified PLD2, is shown to be aldolase C by peptide-mass fingerprinting with matrix-assisted laser desorption/ionization-time-of flight-mass spectrometry (MALDI-TOF-MS). Aldolase A also showed similar binding property as aldolase C and was co-immunoprecipitated with PLD2 in COS-7 cells overexpressing PLD2 and aldolase A. The PH domain corresponding to amino acids 201-310 of PLD2 was necessary for the interaction observed in vitro, and aldolase A was found to interact with the PH domain of PLD2 specifically, but not with other PH domains. PLD2 activity was inhibited by the presence of purified aldolase A in a dose-dependent manner, and the inhibition by 50% was observed by the addition of less than micromolar aldolase A. Moreover, the inclusion of the aldolase metabolites, such as fructose 1,6-bisphosphate (F-1,6-P) or glyceraldehyde 3-phosphate (G-3-P), resulted in an enhanced interaction between PLD2 and aldolase A with a concomitant increase in the potential ability of aldolase A to inhibit PLD2, which suggested the existence of a possible regulation of the interaction by the change of intracellular concentrations of glycolytic metabolites.

PLD2 binding activity with a neuronal protein cytosol, from rat brain, was studied. During the fractionation of rat brain cytosol by four-column chromatography, a 62-kDa PLD2-interacting protein was detected by PLD2 overlay assay and identified as collapsin response mediator protein-2 (CRMP-2), which controls neuronal axon guidance and outgrowth. Using bacterially expressed glutathione S-transferase fusion proteins, we found that two regions (amino acids 65-192 (the phagocytic oxidase domain) and 724-825) of PLD2 and a single region (amino acids 243-300) of CRMP-2 are required for a direct binding of both proteins. A co-immunoprecipitation study in COS-7 cells also showed an in vivo interaction between CRMP-2 and PLD2. Interestingly, CRMP-2 was found to potently inhibit PLD2 activity in a concentration-dependent manner (IC₅₀=30 nM). Over-expression studies also showed that CRMP-2 is an in vivo inhibitor of PLD2 in PC12 cells. Moreover, increasing the concentration of semaphorin 3A, one of the repulsive axon guidance cues, showed that PLD2 activity could be inhibited in PC12 cells. Immunocytochemistry further revealed that PLD2 is co-localized with CRMP-2 in the distal tips of neurites, its possible action site, in differentiated PC12 cells. Taken together, CRMP-2 may interact directly with and inhibit neuronal PLD2, suggesting that this inhibitory mode of regulation may play a role in neuronal pathfinding during the developmental stage.

PLD interacts with PLC-γ1. The epidermal growth factor (EGF) receptor plays an important role in cellular proliferation, and the enzymatic activity of phospholipase C γ1 (PLC-γ1) is regarded to be critical for EGF-induced mitogenesis. PLC-γ1 is co-immunoprecipitated with PLD2 in COS-7 cells. These results of in vitro binding analysis and co-immunoprecipitation analysis in COS-7 cells show that the Src homology (SH) 3 domain of PLC-γ1 binds to the proline-rich motif within the phox homology (PX) domain of PLD2. The interaction between PLC-γ1 and PLD2 is EGF stimulation-dependent and potentiates EGF-induced inositol 1,4,5-trisphosphate (IP3) formation and Ca2+ increase. Mutating Pro-145 and Pro-148 within the PX domain of PLD2 to leucines disrupts the interaction between PLC-γ1 and PLD2 and fails to potentiate EGF-induced IP3 formation and Ca2+ increase. However, neither PLD2 wild type nor PLD2 mutant affects the EGF-induced tyrosine phosphorylation of PLC-γ1. These findings suggest that, upon EGF stimulation, PLC-γ1 directly interacts with PLD2 and this interaction is important for PLC-γ1 activity.

Although the hydrogen peroxide (H2O2)-dependent activation of phospholipase D2 (PLD2) in PC12 cells was previously disclosed, the precise mechanism of PLD1 activation by H2O2 was not revealed (Lee, S. D. et al. (2000) J. Neurochem. 75,1053-1059). In order to find H2O2-dependent PLD2-regulating peptides, the present inventors immunoprecipitated PLD2 from PC12 cells and found that glyceraldehyde 3-phosphate dehydrogenase (GAPDH) co-immunoprecipitated with PLD2 upon H2O2 treatment. This interaction was found to be direct by in vitro reconstitution of purified GAPDH and PLD2. In vitro studies also indicated that PLD2-associated GAPDH was modified on its reactive cysteine residues. Koningic acid, an alkylator of GAPDH on catalytic cysteine residue, also increased interaction between the two peptides in vitro and enhanced PLD2 activity in PC12 cells. Blocking H2O2-dependent modification of GAPDH with 3-aminobenzamide resulted in the inhibition of the GAPDH/PLD2 interaction and attenuated H2O2-induced PLD2 activation in PC12 cells. From the results, we suggest that H2O2 modifies GAPDH on its catalytic cysteine residue not only to inactivate the dehydrogenase activity of GAPDH but also to endow GAPDH with ability to bind PLD2 and resulting association is involved in the regulation of PLD2 activity by H2O2. GAPDH is fundamentally a housekeeping enzyme, involved in cellular energy generation. However, growing evidences have shown that it is not merely a glycolytic enzyme (Sirover, (1999) Biochim. Biophys. Acta 1432, 159-184). It has been shown that GAPDH is involved in the early stage of apoptosis (Berry et al., (2000) J. Neurosci. Res. 60, 150-154). A recent study by Colussi et al. showed that GAPDH inactivation, and hence modification is involved in delaying cellular apoptosis induced by H2O2 (Colussi et al., (2000) FASEB J. 14, 2266-2276). In recent years, it was found that the activation of PLD2 by H2O2 might have anti-apoptotic effect (Lee, S. D. et al. (2000) J. Neurochem. 75, 1053-1059). Based on such studies, it could be speculated that cellular apoptosis mediated by H2O2 could be modulated by GAPDH modification and subsequent interaction and activation of PLD2. And such interaction and PLD activation are related to anti-apoptotic effect in ROS induced apoptosis.

PLD1 interacts with the Akt1. A search of PLD1 binding peptide based on functional relationship and similar cellular location identified Akt1 as a binding peptide. Akt, also known-as PKB (protein kinase B), is a serine/threonine molecule kinase that regulates a variety of cellular molecules implicated in cell proliferation, survival and insulin responses largely by means of phosphorylation. N-terminal pleckstrin homology (PH) domain of Akt is important for activation by virtue of interaction with PI (3,4,5) P3, triggering the targeting to plasma membrane where phosphoinositides are generated (Science. May 31, 2002;296(5573):1655-7. Akt phosphorylates many pro-apoptotic molecules, inhibiting their activity and enhancing cell survival. Akt is also involved in cell cycle progression in G1/S and G2/M transition by phosphorylating cell cycle regulators (J Cell Sci. August 2001;114(Pt 16):2903-10). Amplification of genes encoding Akt isoforms has been found in several types of human cancers (Cell Signal. May 2002;14(5):381-95). The interaction of PLD1 with Akt1 provides yet identified link between PLD1 and the regulation of cell proliferation and survival.

PLD interacts with glucose transporter 4 (GLUT4). Preliminary evidences showed that modulation of PLD peptide was involved in the insulin dependent glucose transporter 4 (GLUT4) translocation in a variety kind of cells. In this study, we found that PLD1 was co-immunoprecipitated with GLUT4 in COS-7 cells overexpressing PLD1 and GLUT4. Thus, we performed pull down assay using glutathione S-transferase fusion peptides corresponding to the cytoplasmic domains of the glucose transporter isoforms with the recombinant PLD1 purified from sf9 cells. PLD1 directly interacted with the central loop of GLUT4 but not with that of GLUT2. The multiple site of PLD1 including N-, C-terminal region was responsible for binding to the central loop of GLUT4. Moreover, in the response to insulin, GLUT4 was co-localized with PLD1 in the plasma membranes of the hlRcB cells overexpressing insulin receptor, however the chimeric GLUT4 which was replaced by the loop region of GLUT2 was not co-localized with PLD1 in the plasma membrane in regardless to insulin. These results suggested that the central loop of GLUT 4 was essential for translocation to the plasma membrane and co-localization with PLD1 in insulin dependent manner.

PLD2 interacts with the mTOR. A search of PLD2 binding peptide based on functional relationship and similar cellular location identified the mTOR (mammalian-target of rapamycin) as a binding peptide (Science. May 14, 1999;284(5417):1161-4, EMBO J. Mar. 1, 2000;19(5):1087-97). mTOR is PIKK-related protein kinase which phosphorylates 4E-BP1 and S6K (Science. Jul. 4, 1997;277(5322):99-101, Proc Natl Acad Sci U S A. Feb. 17, 1998;95(4):1432-7). Activation of 4E-BP1 and S6K is important for translation initiation (Prog Mol Subcell Biol. 2001;26:155-84). Thus, mTOR activity is important for the regulation of cell mass by regulating translation initiation (Genes Dev. Apr. 1, 2001;15(7):807-26). There has not been extensive studies on cellular localization of mTOR, however, it has been reported that it exits in a low-density and cholesterol-enriched membrane fraction. And it is this low-density and cholesterol-enriched membrane fraction in which PLD2 is localized exclusively. Two binding peptides, raptor and GβL, were reported to regulate mTOR activity with different mechanism compared to that of PLD2 (Cell. Jul. 26, 2002;110(2):163-75, Mol Cell. April 2003;11 (4):895-904). The role of FAT domain in mTOR was not revealed but suggested as inhibitory domain for mTOR activity. Binding mapping analysis and translation assay with PLD2 suggest that PLD2 is yet identified regulator of mTOR through complex formation. The identification of PLD2 as negative regulator of mTOR may promote its involvement in translation initiation.

The present inventors investigated PLD2-binding proteins by co-immunoprecipitation assay from PLD2-overexpressed PC12 cells. Protein having a molecular weight of 70 kDa was specifically co-precipitated with PLD2 and identified as Heat Shock Protein (HSP70) using peptide-mass fingerprinting with matrix-assisted laser desorption/ionization (MALDI) mass spectrometer. The amino acid residues 120-428 of HSP70 containing ATPase domain and amino acid residues 186-308 of PLD2 containing PH domain were responsible for the interaction between PLD2 and HSP70. Purified HSP70 potently inhibited PLD2 activity (IC₅₀=200 nM) and the inhibition was dependent on its interaction with PLD2. Furthermore, attenuated activity of PLD2 by HSP70 was stimulated up to 5-fold by ARF without any effect on the interaction between PLD2 and HSP70. These results suggest that PLD2 possesses the potential capacity of ARF responsiveness by the attenuation of high basal activity by HSP70. In response to many metabolic disturbances and injuries including stroke, neurodegenerative disease, epilepsy and trauma, the cell mounts a stress response with induction of a variety of proteins, most notably the 70 kD heat shock protein (Adv Exp Med Biol. 2002; 513: 281-99). Several reports have now shown that selective overexpression of HSPsp70 leads to protection in several different models of nervous system injury. Accordingly, the interaction between PLD and HSPsp 70 and an inhibitor thereof are studied.

The present inventors identified dynamin as a PLD2-interacting peptide in rat brain. Dynamin is a GTPase family member and has been implicated in the formation of nascent vesicles in both the endocytic and secretory pathways. Dynamin was found to interact with PLD2 in a GTP-dependent manner, whereas DYN-K44A, a dominant negative mutant of dynamin, did not interact with PLD2. The PLD2-interacting site on dynamin was identified as the GTPase domain and the dynamin-interacting site on PLD2 overlapped with the PX domain of PLD2. Interaction between dynamin and PLD2 was transiently increased by treatment with EGF in a time dependent manner, and this was found to correlate with the activation of PLD2. Furthermore, the overexpression of DYN-K44A repressed EGF-induced PLD activity and MAP kinase phosphorylation in COS-7 cells. These results suggest that the GTP-dependent interaction of PLD2 with dynamin would likely play an important role in EGF-induced PLD activation and cell signaling. One of the most evident correlation between the severity of dementia due to Alzheimer's disease and its rational cause is the loss of neocortical and hippocampal synapses along with other changes. Axonal terminals are dependent on axoplasmic flow, and that function requires microtubules and the motor proteins kinesin, dynein and dynamin to be intact. (J Neural Transm Suppl. 1998;53:141-5). The present inventors predicted that PLD could interact with dynamin in a GTP-dependent manner. In return, the interaction between Dynamin and PLD may regulate conditions related to Alzheimer's disease.

The present inventors investigated PLD-binding proteins obtained from rat brain extract, and identified a 67-kDa protein as Munc-18-1 by peptide-mass fingerprinting. The direct association between the two proteins was confirmed using purified proteins, and the binding site was determined as the phox homology domain of PLD and multiple sites of Munc-18-1. PLD activity was potently inhibited by Munc-18-1 in vitro. Moreover, the co-transfection of Munc-18-1 and PLD into COS-7 cells was found to inhibit PLD activity, indicating that Munc-18-1 is an intracellular inhibitor of PLD activity. Munc-18-1 was co-precipitated with PLD in a basal state, and PLD2 was colocalized at plasma membrane of COS-7 cells. EGF treatment triggered the dissociation of Munc-18-1 from PLD when EGF activated PLD. Translocation of Munc-18-1 from membrane to cytosol after EGF treatment was also observed by immunocytochemical analysis. The dissociation of Munc-18-1 from the intrinsic PLD and the activation of PLD by EGF were also observed in chromaffin cells. These results suggest that Munc-18-1 is a potent negative regulator of basal PLD activity and that EGF stimulation abolishes their interaction and activates PLD. Munc-18 proteins are related to vesicle trafficking and exocytosis. They are involved in insulin secretion (J Biol Chem. Dec. 29, 2000;275(52):41521-7) and GLUT4 translocation (J Biol Chem. Feb. 9, 2001;276(6):4063-9.). PLD is also involved in these processes. Insulin secretion is known as a determinant of diabetes 1 and GLUT4 translocation is related to diabetes 2. From the results of binding between PLD and Munc-18, it was observed that this interaction is dependent on the extracellular signals and regulates PLD activity. From these results, we can suggest that interaction between PLD and Munc-18-1 can be related in diabetes and by elucidating such activity, we can have a new insight in the specific molecular mechanism of diabetes.

The cholinergic regulation of phospholipase D activity has been widely studied in many cell types. However, whereas activation mechanisms of CCh-induced PLD activation are well known, down regulation mechanisms of PLD activity have not yet been elucidated. To investigate the down regulation mechanism of CCh-induced PLD activation, binding peptides on PLD activity down regulated status were sought. As a result, we found that a 55-kDa molecule in the M3 acetylcoline receptor expressed COS-7 cells, α-, β-tubulin dimmer, specifically interact with PLD2 in time dependent manner. We also observed that tubulin directly interacts with amino acid 65-192 and 475-612 region of PLD and inhibits its activity in vitro. Tubulin constitutes microtubules, a major component of cytoskeleton. Tubulin exists principally in two forms, either as cytosolic soluble tubulin heterodimers consisting of various α- and β-tubulin isotypes or as insoluble assembled tubulin polymers (microtubules). To test the PLD regulation through the changes in dynamics of tubulin, a microtubule structure regulating agent was used. In COS-7 cells, interaction of PLD2 with tubulin increased with nocodazole, a microtubule depolymerizing agent, and decreased with taxol, a microtubule stabilizing agent. In this condition, compared to muscarinic response, nocodazole pretretment inhibits the CCh-induced PLD2 activity while taxol-pretreated cells increased PLD2 activity. Furthermore, in endogeneously muscarinic receptor expressed PC12 cells, a specific and time-dependent association of tubulin with PLD2 was observed after 1 min when the PLD2 activity was in the off-status. And immunocytochemistry further revealed that tubulin translocated to plasma membrane and colocalized with PLD2 after CCh stimulation. Taken together, the results indicate that an increase of monomeric tubulin concentration down regulates carbachol-stimulated PLD activity. Microtubules are involved in many cellular functions such as cell cycle, endocytosis, exocytosis, vesicle trafficking, and maintaining cell shape. Especially, in neuronal cell, microtubule is important for neuronal differentiation, neurite outgrowth, neurite retraction and axon guidance. So, it is possible for this PLD activity regulation by changes in microtubule dynamics to involve these processes. In fact, it is noteworthy that activity shapes the structure of neurons and their circuits. Synaptic activation is shown to produce rapid input-specific changes in dendritic structure (Maletic-Savatic et al., 1999). And the possibility exists that the neurotransmitter-evoked recruitment of tubulin to the membrane assists with this process. In fact, it has been suggested previously that the synaptic activity-controlled balancing of monomeric, dimeric, and polymeric forms of actin and tubulin might underlie the changes in spine shape (Van Rossum and Hanisch, 1999).

PLD interacts with nNOS (neuronal nitric oxide synthase) in rat brain. Co-immunoprecipitation assay with anti-PLD antibody in rat brain was performed and PLD-binding peptide with relative molecular weights of 160 kDa was found. The band was analyzed by matrix-assisted laser desorption/ionization-time-of-flight (MALDI-TOF) mass spectrometry. A search for these masses in a comprehensive sequence database showed that 17 masses (red sequences) matched the calculated tryptic peptide masses of nNOS with an accuracy of <0.1 Da. Nitric oxide (NO), synthesized by the enzyme NO syntase (NOS), is a potent cell signaling and vasodilator molecule that plays important and diverse roles in biological processes including the control of vascular tone and renal sodium excretion. Because of its potent and diverse biological effects, NO production by NOS is under complex and tight control. nNOS can be regulated by interaction with binding partners such as calmodulin, Hsp90, PIN, caveolin-3, PDZ domain containing molecules (PSD-95, PSD-93, 1-syntrophin, CAPON, phosphofurctokinase-M). Calmodulin and Hsp90 activate, and PIN inhibits NOS activity through direct interaction. nNOS more strongly interacts with PLD2 than PLD1 in COS-7 cells. When nNOS was co-immunoprecipited with anti-PLD antibody and PLDs were co-immunoprecipitated with anti-FLAG antibody, nNOS interacts with PLD2 in EGF-dependent manner in COS-7 cells. It was reported that nNOS was stimulated by binding of Ca²⁺-calmoduin according to increased intracellular Ca²⁺ level. It was also reported that EGF can stimulate PLD activity in a time and dose-dependent manner and increase intracellular Ca²⁺ level in COS-7 cells. Therefore, we verified interaction between PLD2 and nNOS after EGF stimulation in COS-7 cells. PLD2 maximally interacts-with nNOS at 0.5-1 min after EGF stimulation.

We showed a specific interaction of PLD with integrin 3, 5 but not 1, both in vitro and in vivo by immunoprecipitation assay. PLD interacts with C-terminal of integrin 5 cytosolic tail. Integrins are alpha-beta heterodimer, and each alpha-beta combination has its own ligand binding specificity and signaling properties. Most integrins recognize several extracellular matrix molecules according to alpha-beta combination. And then, integrin is the place in which several signals are integrated, and involved in many signaling pathway. Many numbers of signaling enzymes and adaptor molecules are regulated by integrin control cell survival, proliferation, motility and differentiation so. It is possible for specific interaction of integrin and PLD to involve in these process especially in cancer and metastasis process.

PLD interacts with GEF-H1. Flag tagged GEF-H1 is coimmunoprecipitated with each of PLD isozymes, especially PLD2 stronger than PLD1, in COS 7 cell. GEF-H1 is a microtubule-interacting peptide, identified by homology to guanine nucleotide exchange factors (GEFs) in a screen of a HeLa cell cDNA library (Ren et al., J Biol Chem, 273(52):34954-60 (1998)). GEF-H1 contains a N-terminal zinc finger domain, a C-terminal coiled-coil domain; immunocytochemistry experiments reveal that these domain are responsible for colocalization of GEF-H1 with microtubules. These recent reports suggested that the GEF activity of GEF-H1 was regulated by association with microtubules (Krendel et al., Nat Cell Biol, Apr;4(4):294-301 (2002)). GEF-H1 also contains a Dbl-type GEF domain, in tandem with a pleckstrin homology domain, a motif typically responsible for exchange of GTP and GDP. GEF-H1 stimulates guanine nucleotide exchange of Rho (known regulators of the cytoskeleton), converting inactive GDP-bound Rho into active GTP-bound form which interacts many effector molecules. Phospholipase D (PLD) is one of those effectors, and plays a role in hydrolysis of phosphatidylcholine to form phosphatidic acid (PA) and choline (Exton, J. H. (1999) Biochim. Biophys. Acta 1439, 121-133). Another postulated role for PLD is in the regulation of the actin cytoskeleton; PLD activity is required for actin cytoskeleton rearrangement to form stress fibers (Exton, J. H., Mol Cell Biol. 2001, 21(12):4055-66).

PLD interacts with V-ATPase subunit A. To investigate another PLD-interacting peptide, we tried to co-immunoprecipiate it with anti-PLD antibody from rat brain cytosol. By molecule peptide-mass fingerprinting, V-ATPase subunit A is identified as a PLD interacting peptide. The vacuolar (H⁺)-ATPase (V-ATPase) is a multisubunit enzyme that facilitates the acidification of intracellular compartments in eukaryotic cells and plays an important role in receptor-mediated endocytosis, renal acidification, bone resorption and activation of acid hydrolases (Forgac, M. (2002) Nat. Rev. Mol. Cell Biol. 3, 94-103). Among the subunits of V-ATPase, subunit A is a 68 kD molecule which has a role in ATP hydrolysis together with another subunit B (Steves, T. H., and Forgac, M. (1997) Annu. Rev. Cell Dev. Biol. 13, 779-808). To confirm the interaction of PLD and V-ATPase subunit A, Flag tagged V-ATPase subunit A is coimmunoprecipitated with each of PLD isozymes, especially PLD2 stronger than PLD1, in COS 7 cell. Since PLD has been implicated in vesicle formation and receptor-mediated endocytosis (Yingjie Shen (2001) Mol Cell Biol. 21(2), 595-602.), PLD might play a new role in regulating receptor endocytosis with the interaction of the V-ATPase.

In terms of domain function of PLD, molecular mechanisms involved in the activity regulation are still unclear. Especially, the involvement of phox homology (PX) domain in targeting and regulation of PLD has not been elucidated. The properties of PLD PX-domains in terms of the phosphoinositide binding and the regulation of PLD activity had been studied. Interestingly, the PX domain of PLD1, but not that of PLD2, specifically interacted with phosphatidylinositols-3,4,5-trisphosphate (Ptdlns(3,4,5)P₃) but not with any other phosphoinositides in both protein-lipid overlay and liposome-binding assays. Mutation of conserved arginine to lysine (R179K) or alanine (R179A) in PX domain of PLD1 disrupted Ptdlns(3,4,5)P₃ binding. EGFP-PLD1 PX domain, but neither the R179K nor R179A mutant, was localized in membrane fraction only when the constitutively active form of phosphoinositide 3-kinase (p110-CAAX) was co-transfected in COS-7 cells, suggesting that the PX domain may interact with Ptdlns(3,4,5)P₃ in cells. PLD1 activity was stimulated by the addition of Ptdlns(3,4,5)P₃ in vitro. The PLD1 mutants (PLD1(R179K) and PLD1(R179A)) were not responsive on Ptdlns(3,4,5)P₃. Co-transfection of p110-CAAX with wild-type PLD1 but not with PLD1(R179K) or PLD1(R179A) significantly increased PLD activity in COS-7 cells. Moreover, insulin-stimulated PLD activity was inhibited by LY 294002, which is a specific inhibitor of phosphoinositide 3-kinase. PLD-induced extracellular signal-regulated kinase (ERK) phosphorylation was observed in wild-type expressing cells but not in cells expressing PLD1(R179K) and PLD1(R179A). Our results suggest that the PLD1 PX domain enables PLD1 to mediate signal transduction through ERK by providing direct binding site for Ptdlns(3,4,5)P₃ and activating PLD1.

PLD plays an important role in the cellular signal transduction and its activity is thought to mediate vesicle trafficking in cells by producing phosphatidic acid (PA), which recruits AP-2, an adaptor protein for clathrin. PLD directly interacts with Na⁺/Cl⁻-dependent dopamine transporter (DAT) which is critical in terminating dopaminergic transmission by removing the transmitter away from the synapse. In human embryonic kidney 293 cells, PLD induces DAT degradation in an activity dependent manner. Furthermore, binding deficient mutant of PLD showed no expression-inhibiting effect on DAT, indicating that activity and direct interaction are important in PLD-mediated down regulation of DAT. Inhibitor studies indicated that PLD shifts the trafficking route of DAT from the steady-state recycling between plasma membrane and endosome to lysosomal degradation pathway. These findings suggest a potential role for PLD activity and interaction in the redistribution and degradation of DAT.

Accordingly, the present invention provides peptide complexes formed between PLD and one or more PLD-interacting peptides selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT). Further, one or more of the interacting molecule members of a peptide complex of the present invention include a variant thereof, a fragment thereof, and a fusion peptide containing such, in addition to a native peptide.

The present invention also provides peptide complexes of the foregoing wherein it comprises a PLD and another peptide selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT).

As described above, individual peptide fragments involved in the specific peptide-peptide interactions have been discovered. -Accordingly, peptide fragments containing the amino acid sequence of the identified regions or variants thereof can be used in forming the peptide complexes of the present invention. In addition, fragments capable of interacting with PLD can also be provided by the combination of molecular engineering of a nucleic acid encoding a PLD-interacting peptide and a method for testing peptide-peptide interaction.

In an embodiment of the peptide complex of the present invention, two or more interacting partners (PLD and one or more peptides selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT), variants thereof, and fragments thereof) are directly fused together, or covalently linked together through a peptide linker, forming a hybrid peptide having a single unbranched polypeptide chain. Thus, the peptide complex may be formed by “intramolecular” interactions between two portions of the hybrid peptide. Again, one or both of the fused or linked interacting partners in this peptide complex may be a native peptide or variants or fragments of a native peptide.

The above-described peptide complexes may further include any additional components, e.g., other peptides, nucleic acids, lipid molecules, monosaccharides or polysaccharides, ions, etc.

The isolated peptide complex of the present invention may be prepared by variety of methods. Specifically, the peptide complex may be isolated directly from an animal tissue sample. Alternatively, the peptide complex may be purified from recombinant host cells that express the members of the peptide complex. As will be apparent to a skilled person in the art, a peptide complex may be prepared from a tissue sample or recombinant host cells by co-immunoprecipitation using an antibody that is immuno-reactive with an interacting peptide partner, or preferably an antibody selectively immuno-reactive with the peptide complex as will be discussed in detail below.

The antibodies can be monoclonal or polyclonal. Co-immunoprecipitation is a commonly used method in the art for isolating or detecting bound peptides. In such procedure, generally a serum sample or tissue or cell lysate is admixed with a suitable antibody. The peptide complex bound to the antibody is precipitated and washed. The bound peptide complexes are then eluted.

Alternatively, immunoaffinity chromatography and immunobloting techniques may also be used to isolate peptide complexes from native tissue samples or recombinant host cells using an immunoreactive antibody with an interacting peptide partner, or preferably an antibody selectively immunoreactive with the peptide complex. Immunoblotting, crude peptide samples from a tissue sample extract or recombinant host cell lysate are fractionated by polyacrylamide gel electrophoresis (PAGE) and then transferred to a membrane, e.g., nitrocellulose. Components of the peptide complex can then be located on the membrane and identified by a variety of techniques, e.g., probing with specific antibodies.

In an embodiment, the peptide complex of the present invention may be prepared from tissue samples or recombinant host cells or other suitable sources by peptide affinity chromatography or affinity blotting. That is, one of the interacting peptide partners is used to isolate the other interacting peptide partner(s) by binding affinity and forming peptide complexes. Thus, an interacting peptide partner prepared by purification from tissue samples or by recombinant expression may be bound covalently or non-covalently to a matrix. The tissue sample extract or cell lysate from the recombinant cells can then be contacted with the bound peptide on the matrix. In affinity blotting, crude peptide samples from the tissue sample or recombinant host cell lysate can be fractionated by polyacrylamide gel electrophoresis (PAGE) and then transferred to a membrane, e.g., nitrocellulose. The purified interacting peptide member is then bound to its interacting peptide partner(s) on the membrane forming peptide complexes, which are then isolated from the membrane.

It will be apparent to one skilled in the art that any recombinant expression methods may be used in the present invention for purposes of expressing the peptide complexes or individual interacting peptides. Generally, a nucleic acid encoding an interacting peptide member can be introduced into a suitable host cell. For purposes of forming a recombinant peptide complex within a host cell, nucleic acids encoding two or more interacting peptide members are introduced into the host cell.

Typically, the nucleic acids, preferably in the form of DNA, are incorporated into a vector to form expression vectors capable of directing the production of the interacting peptide member(s) once introduced into a host cell. Many types of vectors can be used for the present invention.

An epitope tag coding sequence for detection and/or purification of the expressed peptide can also be operably linked to the DNA encoding an interacting peptide member such that a fusion peptide is expressed. Examples of useful epitope tags include, but are not limited to, influenza virus hemagglutinin (HA), Flag, polyhistidine (6× His), GST, and the like. Peptides with polyhistidine tags can be easily detected and/or purified with Ni affinity columns, while specific antibodies immunoreactive with many epitope tags are generally commercially available. The expression vectors may also contain components that direct the expressed peptide in an extracellular or a particular intracellular compartment. Signal peptides, myristoylation signals, palmitoylation signals, and transmembrane sequences are example of optional vector components that can determine the destination of expressed peptides. When it is desirable to express two or more interacting peptide members in a single host cell, the DNA fragments encoding the interacting peptide members may be incorporated into a single vector or different vectors.

The expression vectors can then be introduced into the host cells by any techniques known in the art, e.g., by direct DNA transformation, electroporation, viral infection, lipofection, and the like. The expression of the interacting peptide members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as selection of stable cell lines or site-specific recombination. In stable cell lines, at least the expression cassette portion of the expression vector is integrated into a chromosome of the host cells.

The vector construct can be designed to be suitable for expression in various host cells, including but not limited to bacteria, yeast cells, plant cells, insect cells, and mammalian cells. Methods for preparing expression vectors for expression in different host cells should be apparent to a skilled artisan.

Variants and fragments of the native interacting peptide members can also be easily expressed using the recombinant methods described above. For example, to express a peptide fragment, the DNA fragment incorporated into the expression vector can be selected such that it only encodes the peptide fragment. Likewise, a specific hybrid peptide can be expressed using a recombinant DNA encoding the hybrid peptide. Similarly, a variant peptide may be expressed from a DNA sequence encoding the variant peptide. A variant-encoding DNA sequence may be obtained by manipulating the native peptide-encoding sequence using recombinant DNA techniques. For this purpose, random or site-directed mutagenesis can be conducted using techniques generally known in the art.

To make variants, for example, the amino acid sequence of a native interacting peptide member may be changed in predetermined manners by site-directed DNA mutagenesis to create or remove consensus sequences for, e.g., phosphorylation by protein kinases, glycosylation, ribosylation, myristolation, palmytoylation, ubiquitination, and the like. Alternatively, non-natural amino acids can be incorporated into an interacting peptide member during the synthesis of the peptide in recombinant host cells. In addition, variants of the native interacting peptide members of the present invention can also be prepared by chemically linking certain moieties to amino acid side chains of the native peptides. If desired, variants thus generated can be tested to determine whether they are capable of interacting with their intended partners to form peptide complexes. Testing can be conducted by other methods known in the art for detecting peptide-peptide interaction.

A hybrid peptide as described above having PLD or a variant or fragment thereof covalently linked by a peptide bond or a peptide linker to a peptide selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp7o), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT), a variant thereof, and a fragment thereof, can be expressed recombinantly from a chimeric nucleic acid, e.g., a DNA or mRNA fragment encoding the fusion peptide.

The modulators selected in accordance with the screening method of the present invention can be effective in modulating the functions or activities of PLD, a PLD-interacting peptide, or the peptide complexes of the present invention. Any test compounds may be screened by the screening method of the present invention to select modulators of PLD, a PLD-containing peptide complex and/or a PLD-interacting peptide of the present invention.

The test compounds may be screened to identify compounds capable of binding the peptide complexes or interacting peptide members thereof in accordance with the present invention. For this purpose, a test compound is contacted with a peptide complex or an interacting peptide member thereof under conditions and for a time sufficient to allow specific interaction between the test compound and the target components to occur and thus binding of the compound to the target forms a complex. Subsequently, the binding event is detected.

Various screening techniques known in the art may be used in the present invention. The peptide complexes and the interacting peptide members thereof may be prepared by any suitable methods, e.g., by recombinant expression and purification. The peptide complexes and/or interacting peptide members thereof (both are referred to as “target” hereinafter in this section) may be free in solution. A test compound may be mixed with a target, to form a liquid mixture. The compound may be labeled with a detectable marker. Upon mixing under suitable conditions, the binding complex of the compound and the target may be co-immunoprecipitated and washed. The compound in the precipitated complex may be detected based on the marker on the compound.

In an embodiment, a peptide complex used in the screening method includes a fusion peptide, which is formed by fusion of two interacting peptide members or fragments or interaction domains thereof. The fusion peptide may also be designed such that it contains a detectable epitope tag fused thereto. Suitable examples of such epitope tags include sequences derived from, e.g., influenza virus hemagglutinin (HA), polyhistidine (6.times.His), c-myc, MBP, GST, and the like.

Test compounds may be also screened to identify compounds capable of dissociating the peptide complexes identified in accordance with the present invention. Thus, for example, a PLD-containing peptide complex can be contacted with a test compound and the peptide complex can be detected. Conversely, test compounds may also be screened to identify compounds capable of enhancing the interaction between PLD and a PLD-interacting peptide or stabilizing the peptide complex formed by the two or more peptides. The method of screening would be carried out in a similar manner as described above. For example, the presence of a particular peptide complex can be detected by an antibody selectively immunoreactive with the peptide complex. Thus, after incubation of the peptide complex with a test compound, an immunoprecipitation assay can be conducted with the antibody. If the test compound disrupts the peptide complex, then the amount of immunoprecipitated peptide complex in this assay will be significantly less than that in a control assay in which the same peptide complex is not contacted with the test compound. Similarly, the interaction between two peptides, which is to be enhanced, may be incubated together with the test compound. Thereafter, the peptide complex may be detected by the selectively immunoreactive antibody. The amount of peptide complex may be compared to that formed in the absence of the test compound. Various other detection methods may be suitable in the dissociation method, as will be apparent to one skilled in the art.

The screening methods of modulators may be carried out both in vivo and in vitro environments. For example, any in vivo or in vitro assays known in the art to be useful in identifying modulators capable of strengthening or interfering with the stability of the peptide complexes of the-present invention may be used.

The screening assays of the present invention are useful in identifying compounds capable of interfering with or disrupting or dissociating peptide-peptide interactions between PLD, or a variant thereof, and a peptide selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) and a variant thereof. For example, PLD, or a variant thereof, and its interacting partners, or variants thereof, are believed to play a role in budding, intracellular vesicle trafficking and vacuolar peptide sorting, formation of multivesicular bodies, exocytosis, endocytosis, tumorigenesis and cell transformation, and autoimmune response, neuronal disorder and thus are involved in cancer and autoimmune diseases. It may be possible to ameliorate or alleviate the diseases or disorders in a patient by interfering with or dissociating normal interactions between PLD and one of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT). Alternatively, if the disease or disorder is associated with increased expression of PLD and/or one of the PLD-interacting peptides in accordance with the present invention, then the disease may be treated or prevented by weakening or dissociating the interaction between PLD and the PLD-interacting peptide in patients. In addition, if a disease or disorder is associated with variants of PLD and/or one of the PLD-interacting peptides that lead to strengthened peptide-peptide interaction therebetween, then the disease or disorder may be treated with a compound that weakens or interferes with the interaction between the variant of PLD and/or the PLD-interacting peptide(s).

In a screening method for modulators of an interaction, PLD (or fragment thereof, or a variant of PLD (or fragment thereof, and a PLD-interacting peptide (or a variant or fragment thereof, or a variant of a PLD-interacting peptide (or fragment thereof), are used as test peptides expressed in the form of fusion peptides as described above for purposes of checking interaction of PLD and PLD-interacting peptide.

In an embodiment, a counterselectable marker is used as a reporter such that a detectable signal (e.g., appearance of color or fluorescence) is present only when the test compound is capable of interfering with the interaction between the two test peptides.

In another embodiment, the interaction or interaction change between the first peptide and the second peptide is determined in a host cell.

The screening assays of the present invention can also be used in identifying compounds that trigger or initiate, enhance or stabilize peptide-peptide interactions between PLD, or a variant thereof, and a peptide selected from the group consisting of actin, aidoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT), and a variant thereof. For example, if a disease or disorder is associated with decreased expression of PLD and/or a member of selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT), then the disease or disorder may be treated or prevented by strengthening or stabilizing the interaction between PLD and the PLD-interacting member in patients. Alternatively, if a disease or disorder is associated with variant of PLD and/or variants of a PLD-interacting peptide that lead to weakened or abolished peptide-peptide interaction therebetween, then the disease or disorder may be treated with a compound that initiates or stabilizes the interaction between the variants of PLD and/or the variants of PLD-interacting peptide(s).

Thus, a screening assay can be performed in the same manner as described above, except that a positively selectable marker is used. For example, PLD (or a variant or fragment thereof), or a variant of PLD (or a variant or fragment thereof), and a peptide selected from the group consisting-of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) (or a variant or fragment thereof), or a variant of a peptide selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) (or a variant or fragment thereof), are used as test peptides expressed in the form of fusion peptides as described above for purposes of pull down assay. The fusion peptides are expressed in host cells and are allowed to interact with each other in the presence of one or more test compounds.

Generally, a control assay is performed in which the above screening assay is conducted in the absence of the test compound. The result is then compared with that obtained in the presence of the test compound.

In an embodiment, the screening method further comprises generating a data set defining one or more selected test compounds, wherein the data set is embodied in a transmittable form.

As described above, the interactions between PLD and the PLD-interacting peptides suggest that these peptides and/or the peptide complexes may be involved in common biological processes and disease pathways. Thus, one may modulate such biological processes or treat diseases by modulating the functions and activities of PLD, a PLD-interacting peptide, and/or a peptide complex comprising a combination of these peptides. As used herein, modulating PLD, a PLD-interacting peptide, or a peptide complex comprising a combination of these peptides means altering (enhancing or reducing) the activities of the peptides or peptide complexes, e.g., increasing the concentrations of PLD, a PLD-interacting peptide or a peptide complex comprising a combination of these peptides, enhancing or reducing their biological activities, increasing or decreasing their stability, altering their affinity or specificity to certain other biological peptides; etc. For example, a PLD-containing peptide complex of the present invention or its members thereof may be involved in budding, intracellular vesicle trafficking and vacuolar peptide sorting, formation of multivesicular bodies, endocytosis, tumorigenesis and cell transformation, and proliferation and may be associated with diseases and disorders such as neurodegenerative diseases, cancer and diabetes, and the present invention may be used in determining the effect of an aberration in a particular PLD-containing complex or an interacting member thereof on the above specified occurrences. In addition, it is also possible to determine, using the same assay methods, the presence or absence of an association between a PLD-containing complex or an interacting member thereof and a physiological disorder or disease such as cancer and autoimmune diseases or predisposition to a physiological disorder or disease.

In accordance with this aspect of the present invention, methods are provided for modulating (promoting or inhibiting) a PLD-containing protein complex or interacting member thereof. The human cells can be in in vitro cell or tissue cultures.

In one embodiment, the concentration of a PLD-containing protein complex of the present invention is reduced in the cells. Various methods can be employed to reduce the concentration of the protein complex. The protein complex concentration can be reduced by interfering with the interaction between the interacting members. For example, compounds capable of interfering with interactions between PLD and a protein selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) can be administered to the cells in vitro or in vivo. Such compounds can be compounds capable of binding PLD or the protein selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT). They can also be antibodies immunoreactive with the PLD or the protein selected from the group consisting of actin, aldoalse, collapsin response mediator peptide-2-(CRMP-2), phospholipase C-γ1 (PLC-γ₁), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT). Also, the compounds can be small peptides derived from a PLD-interacting protein or mimetics thereof capable of binding PLD, or small peptides derived from TPLD protein or mimetics thereof capable of binding a protein selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT).

In various embodiments described above, preferably, the concentrations or activities of both PLD protein and a PLD-interacting protein are reduced or inhibited.

In yet another embodiment, an antibody selectively immunoreactive with a protein complex having PLD interacting with a protein selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT) is administered to cells in vitro or in human bodies to inhibit the protein complex activities and/or reduce the concentration of the protein complex in the cells or patient.

In one aspect of the present invention, methods are provided for reducing in cells or tissue the concentration and/or activity of a protein complex identified in accordance with the present invention that comprises PLD and one or more members of the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1-(GEF-H1), V-ATPase, phosphoinositide-3-phosphate (PIP3), dopamine transporter (DAT). In addition, methods are also provided for reducing in cells or tissue the concentration and/or activity of a PLD-interacting protein selected from the group consisting of actin, aldoalse, collapsin response mediator peptide-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT). By reducing the concentration of protein complex and/or the PLD-interacting protein concentration(s) and/or inhibiting the functional activities of the protein complex and/or the PLD-interacting protein(s), the diseases involving such protein complex or PLD-interacting protein(s) may be treated or prevented.

The present invention is further illustrated and described by the following examples, which should not be taken to limit the scope of the invention.

### EXAMPLE 1

### Interaction between PLD2 and β-actin

(1) Co-Precipitation of PLD-2-Binding Proteins from Rat Brain Extracts

Hexa-histidine (His₆)-tagged PLD2 was purified from detergent extracts of baculovirus-infected sf9 cells by chelating-Sepharose affinity column chromatography according to J. H. Kim et. al., FEBS Lett. 454, 42-46,1999. Rat brains (3 g) were homogenized in homogenation buffer (20 mM Tris/HCl, pH 7.5,1 mM MgCl₂, 1 mM EDTA, 1 mM EGTA, and 150 mM NaCl) using a polytron homogenizer. After centrifugation at 100,000×g for 1 hour at 4° C., the resulting supernatant was used to investigate potential PLD2-binding partners. Protein concentrations in the brain extract were determined using the method according to M. M. Bradford, Anal. Biochem. 72, 248-254, 1976.

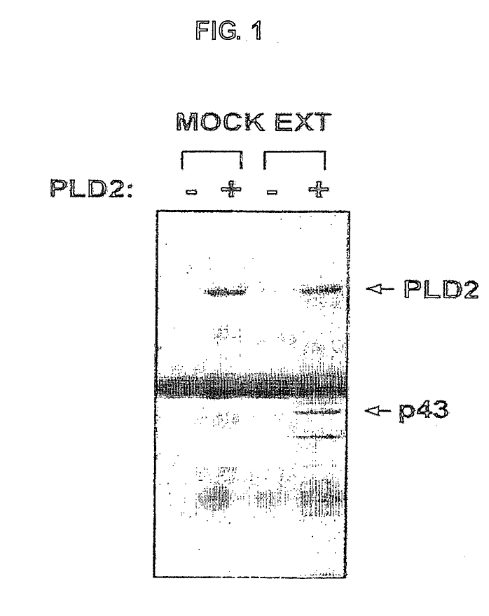

Affinity-purified anti-PLD antibodies immobilized on protein A resin (PLD antibody complex) were incubated with purified recombinant PLD2 (3 μg) for 2 hours. After a brief centrifugation, the immune complexes were washed three times with radioimmune precipitation buffer (50 mM Tris/HCl, pH 8.5, 0.1% SDS, 150 mM NaCl, 1% Triton X-100, and 1% deoxycholate). The prepared brain extract (3 mg of protein) was then incubated with the complexes for 2 hours at 4° C. Finally, the co-precipitated proteins were washed again three times with radioimmune precipitation buffer, loaded onto a gel, and visualized by Coomassie Brilliant Blue staining. The result was shown in FIG. 1, which shows anti-PLD antibody complexes in the absence (−) or presence (+) of recombinant PLD2 incubated with homogenation buffer (MOCK) or rat brain extract (EXT). As indicated by an arrow in FIG. 1, a 43-kDa protein is detected as a PLD2-binding protein.

(2) Identification of the 43-kDa Protein

Identification of the 43-kDa protein obtained in the above was performed using peptide mass fingerprinting by matrix-assisted laser desorption ionization/time-of-flight mass spectrometry, according to J. B. Park et. al., J. Biol. Chem. 275, 21295-21301, 2000. The fraction containing the 43-kDa protein (p43) after co-immunoprecipitation from rat brain extract was separated by 8% SDS-PAGE, and the band corresponding to p43 was excised and digested with trypsin (Roche Molecular Biochemicals) for 6 hours at 37° C. The masses of the tryptic peptides obtained were determined with a Voyager DE time-of-flight mass spectrometer (Perceptive Biosystems, Inc., Framingham, Mass.) in the Korea Basic Science Institute (Busan, Korea). The masses obtained, marked as P1-P7, were compared with protein in the Swiss-Protein database using the MS-Fit peptide mass search program. As shown in Table 1, the peptide exhibited molecular masses that were almost identical to the calculated masses of the corresponding theoretically predicted tryptic peptides of β-actin.

TABLE 1

| | | M + H⁺ | |
|---|---|---|---|
| | | Observed | Calculated^{b} |
| Peptide | Sequence^{a} | Da | |
| P1 | LDLAGR (178-183) | 644.36 | 644.37 |
| P2 | ILAPPER (329-335) | 795.49 | 795.47 |
| P3 | GYSFTTTAER (197-206) | 1132.54 | 1132.52 |
| P4 | HQGVMVGMGQK (40-50) | 1187.57 | 1187.56 |
| P5 | QEYDESGPSIVHR (360-372) | 1516.72 | 1516.70 |
| P6 | SYELPDGQVITIGNER (239-254) | 1790.90 | 1790.89 |
| P7 | VAPEEHPVLLTEAPLNPK | 1954.03 | 1954.06 |
| | (96-113) | | |

^{a}The matched peptides cover 21% (81 of 375 amino acids) of the proteins
^{b}Monoisotopic mass

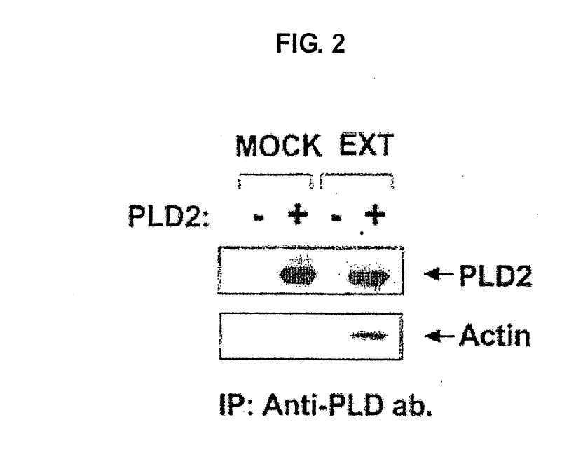

To substantiate the identity of this protein further, the presence of actin in the PLD2 precipitate was confirmed using a monoclonal antibody to actin. As shown in FIG. 2, actin was strongly detected in the PLD2 precipitate but not in a control immune complex. On the basis of these results, it is concluded that the 43-kDa protein in the PLD2 precipitate from the rat brain extract is β-actin.

### EXAMPLE 2

### Interaction between PLD2 and Aldolase

(1) 40 kDa Protein from Rat Brain was Detected as a PLD2-Direct Binder Using Blot Overlay Assay.

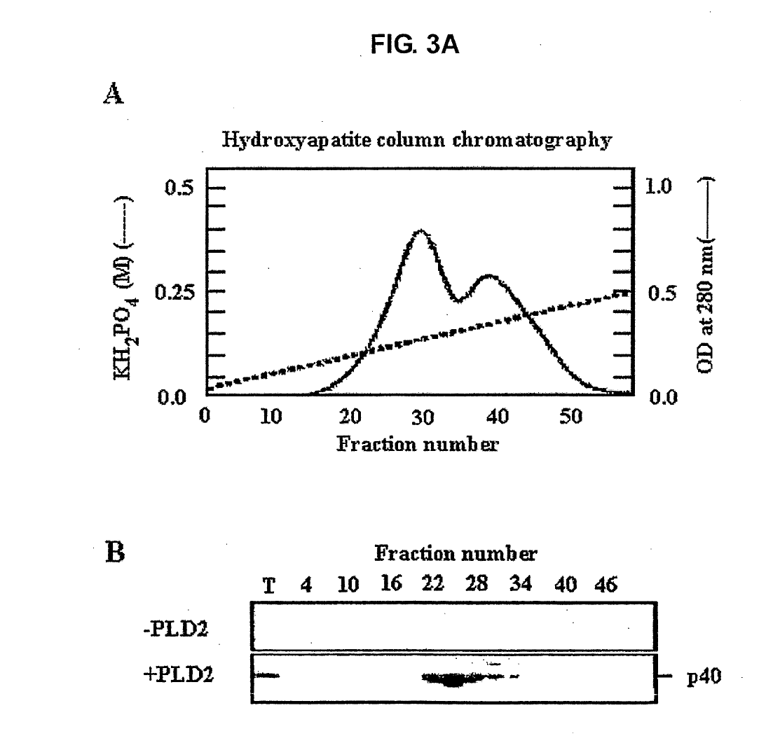

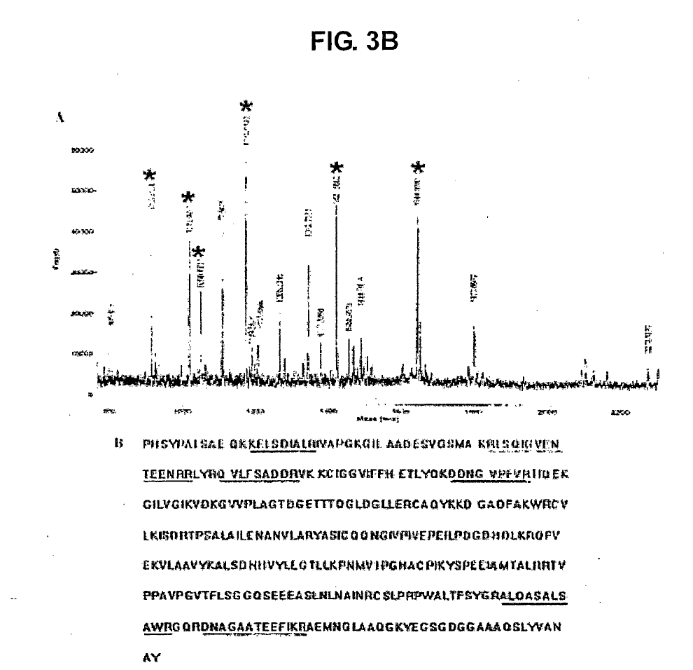

All preparations were performed at 4° C. or on ice. Adult rat brains (total 30 g) were homogenized using a polytron homogenizer in homogenation buffer containing 20 mM Tris, pH 7.6, 1 mM MgCl₂, 1 mM PMSF, and 0.1 mM DTT. The homogenate was centrifuged at 100,000 g for 1 h and the resulting supernatant (the cytosolic fraction) was collected. The cytosolic fraction (900 mg) was loaded to a Q-Sepharose anion exchange column (13 cm×3 cm) pre-equilibrated with buffer A (20 mM Tris, pH 7.5, 1 mM EDTA, 1 mM EGTA, 1 mM MgCl₂, and 0.1 mM DTT). Unbound proteins (flow-through fractions) were collected, and NaCl was added thereto to 2 M. After centrifugation (50,000g, 20 min), the resulting supernatant was loaded onto a Phenyl Sepharose column (70 cm×2 cm). Proteins were eluted at a flow rate of 2 mL/min by applying a decreasing gradient of NaCl (from 2 M to 0 M) over a period of 60 min. Fractions were collected and tested by blot overlay assay with purified PLD2. Peak fractions were pooled, and diluted with buffer A to adjust the salt concentration to 50 mM NaCl, and then loaded onto a Hi-Trap heparin column (1 mL, Pharmacia) pre-equilibrated with buffer A containing 50 mM NaCl. Bound proteins were then eluted at a flow rate of 0.5 mL/min using a linear gradient of 0.05-1 M NaCl over 30 min. Fractions were collected and tested by blot overlay assay. Fractions containing PLD2-interacting proteins were pooled and continuously loaded onto a Bio Gel HT (1 mL, Bio-Rad) pre-equilibrated with buffer B (20 mM Tris, pH 7.6, 50 mM NaCl, 0.1 mM DTT). Bound proteins were eluted at a flow rate of 0.3 mL/min by applying an increasing gradient of 0-0.25 M KH₂PO₄. Then, fractions were collected and tested using the blot overlay assay with purified PLD2. PLD2 overlay assay was performed as previously described in J. B. Park et. al., J. Biol. Chem. 275, 21295-21301, 2000. In brief, rat brain cytosolic proteins were separated by SDS-PAGE and transferred to nitrocellulose membranes. The blots were preincubated overnight with PLD assay buffer (50 mM HEPES, pH 7.3, 3 mM EGTA, 3 mM CaCl₂, 3 mM MgCl₂, 80 mM KC1) containing 0.1 mM DTT and 5% (w/v) skim milk at room temperature and then incubated with the same buffer containing 1 μg/mL of purified PLD2 for 3 h at room temperature. The membranes were washed several times with Tris-buffered saline (20 mM Tris-HCl, pH 7.5, 150 mM NaCl) containing 0.05% Tween 20 and reacted with polyclonal antibodies directed against PLD for 3 h. After the washings, the membranes were subsequently incubated with horseradish peroxidase-conjugated goat anti-rabbit IgG antibodies for 1 h and developed using an enhanced chemiluminescence kit as described by the manufacturer. The technique used has been previously described in J. B. Park et. al., J Biol. Chem. 275, 21295-21301, 2000. In brief, the fraction containing the 40 kDa protein (p40) obtained by hydroxyapatite column chromatography was separated by 10% SDS-PAGE, and the band corresponding to p40 was excised and digested with trypsin (Roche Molecular Biochemicals) for 6 h at 37° C. FIG. 3A shows the isolation of PLD2-binding protein (p40) by the above blot overlay assay.

The masses of the tryptic peptides so obtained were determined using a Bruker REFLEX reflector time-of-flight mass spectrometer (Bruker-Franzen, Bremen, Germany). Delayed ion extraction resulted in peptide masses in 50 ppm mass accuracy or higher on average. Using the amino acid sequences and the mass numbers of the tryptic peptides of p40, the Swiss-Protein database was searched for a protein match. FIG. 3B shows the matrix-assisted laser desorption ionization mass spectrum of the digested peptides of p40. The masses obtained were compared with protein in the Swiss-Protein database using the MS-Fit peptide mass search program. The peptide exhibited molecular masses that were almost identical to the calculated masses of the corresponding theoretically predicted trytic peptides of aldolase.

(2) Association between PLD2 and Aldolase.

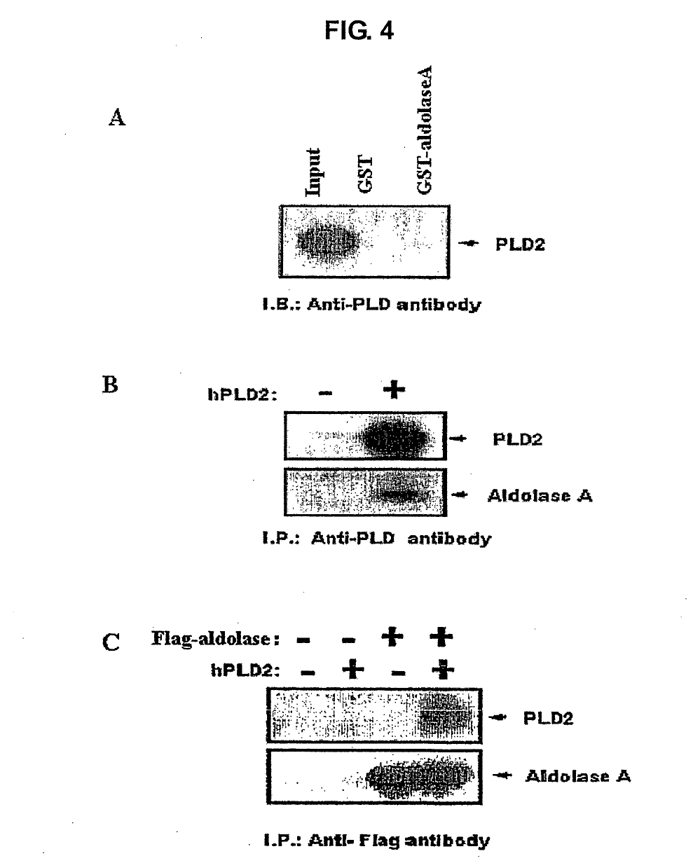

In vitro binding between all the GST fusion proteins and aldolase was performed in PLD assay buffer (50 mM HEPES/NaOH, pH 7.3, 3 mM EGTA, 3 mM CaCl₂, 3 mM MgCl₂, 80 mM KCl) containing 1% Triton X-1 00 for 1 h at 4° C. After a brief centrifugation, the precipitated complexes were washed 3 times with the same buffer before being loaded onto a polyacrylamide gel. COS-7 cells were transfected in combination with pCDNA 3.1 vector harboring human PLD2 and pCMV vector containing aldolase A. The cultured cells were harvested and lysed with PLD assay buffer containing 1% Triton X-100, 1% cholate, and 1 mM PMSF. After a brief sonication, the lysates-were-centrifuged at 100,000 g for 30 min and the cell extracts (1 mg) were incubated respectively with anti-PLD or anit-flag antibody-conjugated protein A Sepharose overnight. After a brief centrifugation, the co-immunoprecipitated complexes were washed 3 times with the same buffer before being loaded onto a gel. As shown in FIG. 4, aldolase strongly interacts with PLD2.

### EXAMPLE 3

### Interaction between PLD2 and CRMP

(1) Identification of p62 as CRMP-2.

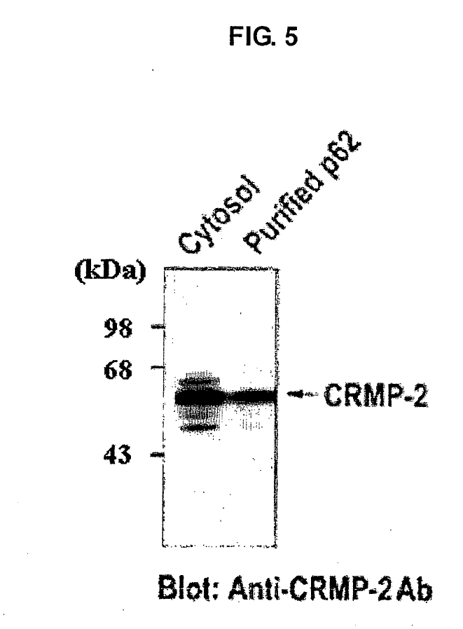

Purified PLD₂-interacting protein from the hydroxylapatite column in Example 2 was digested for 2 h at 37° C. with V8 protease obtained from Staphylococcus aureus and then subjected to 15% SDS-PAGE to separate the cleaved peptides. After transferring the peptides to a polyvinylidene difluoride membrane, they were stained with Coomassie Brilliant Blue, rinsed several times with 30% methanol, excised, and subjected to Edman degradation. The candidate protein was identified by sequencing (ABI473 Sequencer) at the Institute of Basic Science (Busan, Korea) and by comparing the results obtained from the Swiss-Protein database using the BlastP algorithm. The masses obtained were compared with protein in the Swiss-Protein database using the MS-Fit peptide mass search program. The peptide exhibited molecular masses that were almost identical to the calculated masses of the corresponding theoretically predicted tryptic peptides of CRMP-2 (see FIG. 5).

(2) Direct Interaction of CRMP-2 with PLD2 in vitro.

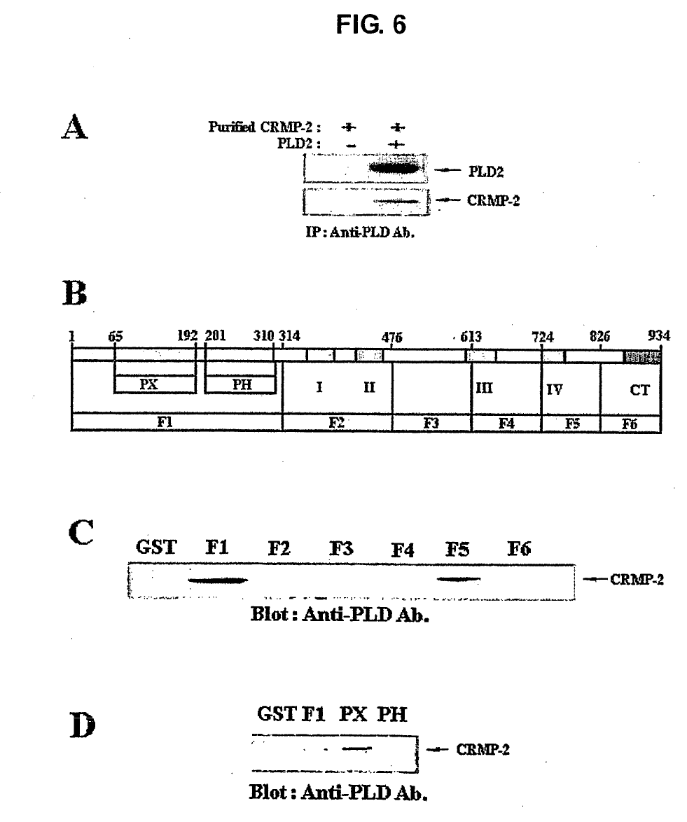

Affinity-purified anti-PLD2 antibodies immobilized with protein A beads were first incubated with purified PLD2 for 2 h at4° C. After a brief centrifugation, the precipitates were reincubated with the indicated amounts of purified CRMP-2 for 15 min at 37° C. in PLD assay buffer containing 1% Triton X-100. Binding site mapping between PLD2 and CRMP-2 was performed by incubating the indicated amounts of glutathione S-transferase (GST) fusion proteins with purified PLD2 or rat CRMP-2, respectively, under the same buffer conditions for 15 min at 37° C. After a brief centrifugation, the pellets were washed three times with the same buffer before being loaded onto a polyacrylamide gel. As shown in FIG. 6, CRMP-2 strongly interacts with PLD2.

### EXAMPLE 4

### Interaction between PLD2 and PLC-γ1

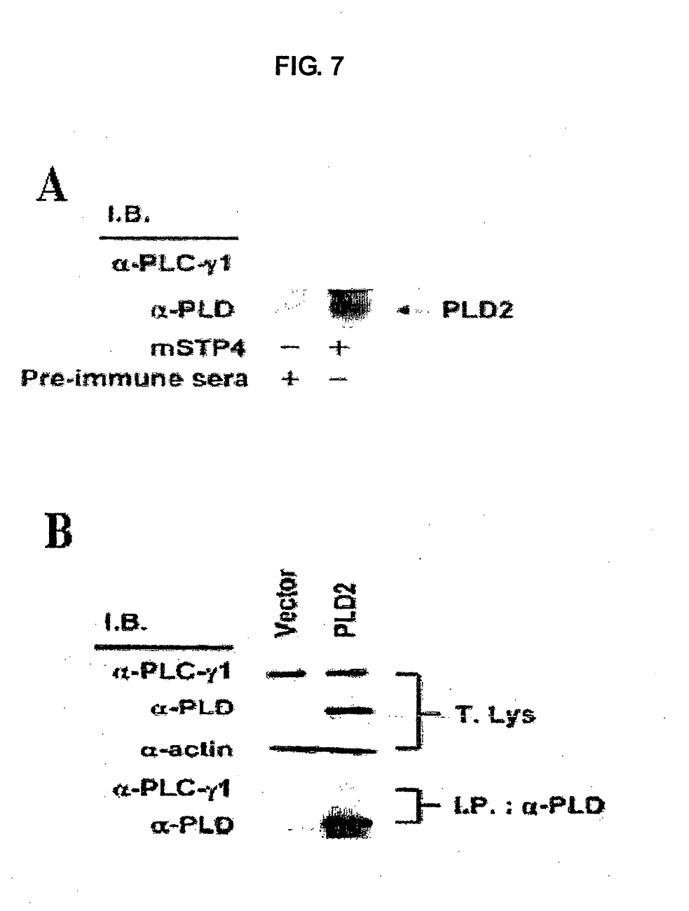

Recombinant rat PLC-β1, PLC-γ1, and human PLD2 were expressed in Sf9 cells and purified. Cells were lysed with IP buffer (20 mM Tris/HCl, pH 8.0, 150 mM NaCl, 1 mM MgCl₂, 1 mM EGTA, 1% Triton X-100, 1% cholic acid) by sonication. Cell lysates were centrifuged at 100,000×g at 4° C. for 30 min, and the supernatants were incubated with an antibody immobilized to Protein A-Sepharose beads. In vitro binding was performed in 300 μl of binding buffer (20 mM Tris/HCl, pH 8.0, 150 mM NaCl, 1 mM MgCl₂, 1 mM EGTA, 0.1% Triton X-100) at 4° C. for 3 h. Proteins were denatured by boiling for 5 min at 95° C. in a Laemmli sample buffer, separated by SDS-PAGE, and immunoblot analysis was performed as described previously. As shown in FIG. 7, PLC-γ1 strongly interacts with PLD2.

### EXAMPLE 5

### Interaction between PLD2 and GAPDH

(1) H₂O₂-Dependent Association of p35 to PLD2 in PC12 Cells.

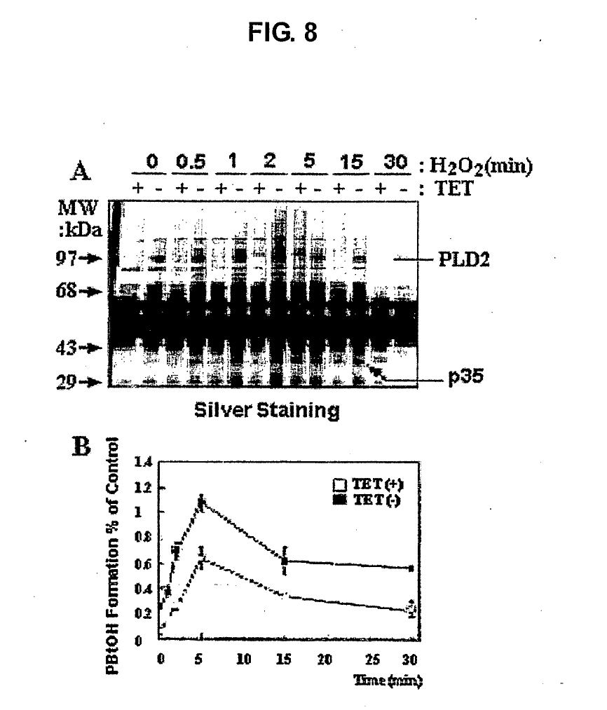

PLD2-inducible PC12 cells were subcultured in the presence or absence of 0.5 μg/mL tetracycline for 24 h. After additional 24 h of serum deprivation, cells were treated with H₂O₂ or KA and washed with ice-cold buffer A (50 mM HEPES/NaOH, pH 7.5, 80 mM KCl, 2.5 mM MgCl₂, 3 mM EGTA) containing protease inhibitors twice and harvested by scraping. Inhibitors were pre-incubated when required and included during incubation periods. Cells were lysed with buffer A containing 1% n-octyl-β-D-glucopyranoside and protease inhibitors by brief sonication. Soluble extracts were obtained by centrifugation for 1 h, 100,000×g at 4° C. Resulting supernatants were incubated with anti-PLD antibody (5 μg) coupled to Protein A Sepharose bead at 4° C. After 5 h of incubation, resulting pellets were washed four times with buffer A containing 1% n-octyl-β-D-glucopyranoside before being separated by sodium dodecyl sulfate-polyacrylamide gel electrophoresis and silver staining or immunoblotting. As indicated by an arrow in FIG. 8, a 35-kDa protein is detected as a PLD2-binding protein.

(2) Identification of p35 as Glyceraldehyde 3-Phosphate Dehydrogenase.

Protein band excised from gels were subjected to in-gel digestion with trypsin following the method reported after intensive destaining with 40% methanol (Jensen et aL 1999). Spectra were obtained using a Bruker Reflex III matrix assisted laser desorption/ionization time-of-flight mass spectrometer. Tryptic peptides were desalted and concentrated with homemade nano-scale reverse-phase columns using GeLoader tip as described previously (Gobom et al. 1999). Peptide mixtures were loaded directly to MALDI probes using a 20 mg/mL alpha-cyano-4-hydroxycinnamic acid (Sigma) as a matrix. Spectra were calibrated internally using trypsin autodigestion products (m/z 842.51 and 2211.10). The search program ProFound (http://www.proteometrics.com) was used for searches in the database NCBlnr (Zhang and Chait 2000). The matrix-assisted laser desorption ionization mass spectrum of the digested peptide of p35 exhibits molecular masses almost identical to the calculated masses of the corresponding theoretically predicted tryptic peptides of that was glyceraldehyde 3-phosphate dehydrogenase.

### EXAMPLE 6

### Interaction between PLD2 and Akt1

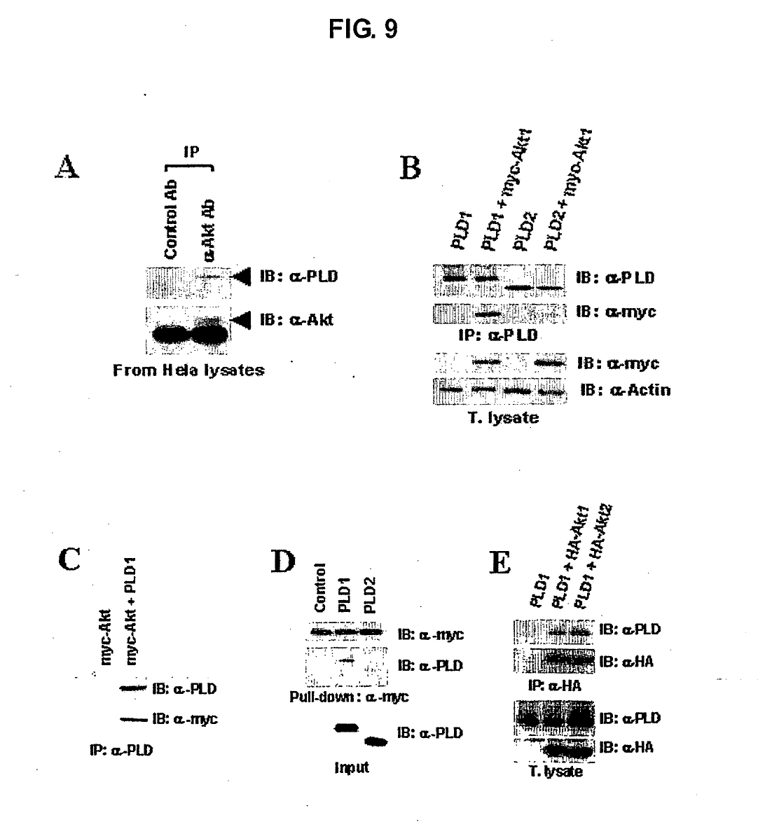

COS7 cells in serum growing states were lysed with Buffer B containing 1% cholic acid, 1% Triton X-100, protease inhibitor and phosphatase inhibitor cocktail. After extensive sonication, lysates were clarified by centrifugation at 15,000 rpm. 1.5 mg of lysates were incubated with either anti-PLD antibody or control antibody, precoupled to Protein A agarose bead, for over 4 hrs at 4° C. After being washed with Buffer B five times, the complexes were subjected to immunoblot analysis and the existence of Akt was revealed by specific anti-Akt monoclonal antibody. COS 7 cells were cotransfected with either PLD1 or PLD2 with wild-type Akt as indicated. Cells were lysed and sonicated in Buffer B containing 1% cholic acid and 1% Triton X-100. After immunoprecipitation with anti-PLD antibody, followed by washing three times, the amount of proteins in precipitates was revealed by indicated antibody. Results were reproduced in two dependent experiments. Akt (100 ng) purified from baculo virus-infected Sf9 cells was incubated in the absence (−) or presence (+) of purified PLD1 for 30min at 4 ° C. Akt/PLD1 complex was isolated by anti-PLD antibody precoupled onto Protein A agarose bead for another 30 min at 4° C. After being washed with buffer A containing 0.5% Triton X-100 and 0.3% BSA three times, the complexes were subjected to immunoblot analysis using anti-PLD antibody and anti-myc antibody. Prebound Akt onto anti-myc antibody was incubated with either purified PLD1 or PLD2 for 1 hr at 4° C. After washing with buffer A containing 0.5% Triton X-100 and 0.3% BSA three times, the complexes were subjected to immunoblot analysis and the amount of coprecipitated Akt was revealed by anti-myc antibody. All the results shown are representative of two independent experiments. As shown in FIG. 9, PLD1 forms peptide complexes with Akt strongly and specifically both in vitro and in vivo.

### EXAMPLE 7

### Interaction between PLD2 and Glucose Transporter 4

(1) Association with PLD1 and GLUT4.

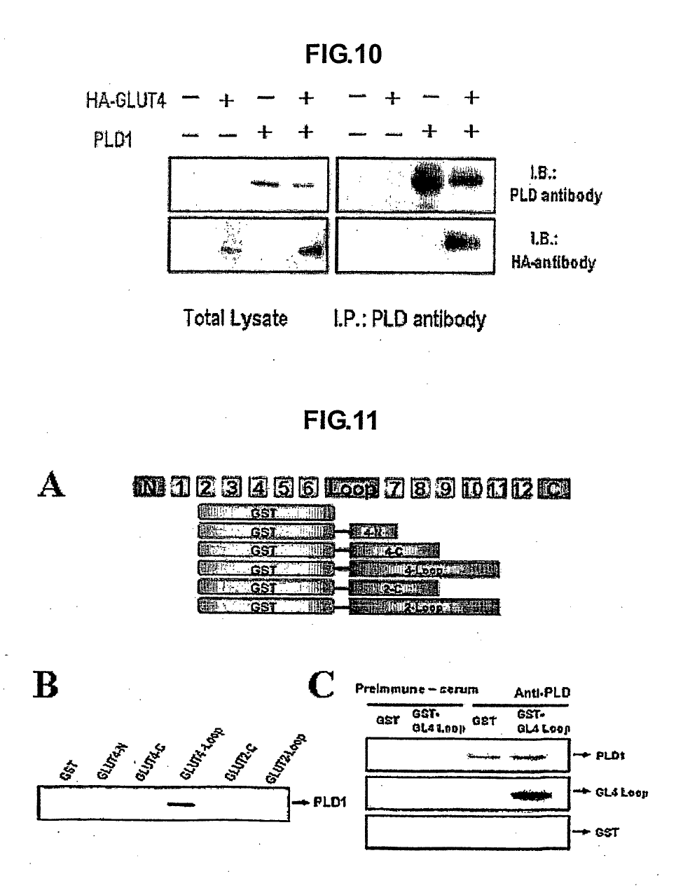

COS-7 cells were transfected in combination with pcDNA 3.1 vector harboring PLD1 and HA-GLUT4. The cultured cells were harvested and lysed with PLD assay buffer containing 1% Triton X-100, 1% cholate, and 1 mM PMSF. After a brief sonication, the lysates were centrifuged at 100,000×g for 30 min and the cell extracts (2 mg) were incubated with anti-PLD body-conjugated protein A Sepharose for 4 hrs. After a brief centrifugation, the co-immunoprecipitated complexes were washed three times with the same buffer before being loaded onto a gel. FIG. 10 shows association with PLD1 and GLUT4.

(2) Direct Binding of Purified PLD1 to the Cytoplasmic Central Loop of GLUT4.

All experiments were performed at 4° C. on ice. In vitro binding between the GST-fusion proteins corresponding to N, C-termius and central loop of GLUT and PLD1 and between GST-fragments of PLD1 and MBP-fusion central loop of GLUT4 were performed in the PLD assay buffer (50 mM Hepes/NaOH, pH 7.4, 3 mM EGTA, 3 mM CaCl₂.3 mM MgCl₂, 80 mM KCl) containing 1% Triton X-100 and 5% glycerol for 1.5 hrs at 4° C. After a brief centrifugation, the precipitated complexes were washed three times with the same buffer before being loaded onto a polyacrylamide gel. Affinity-purified anti-PLD1 antibody immobilized with protein A beads was first incubated with purified PLD1 for 2 h at 4° C. After washing, the precipitates were reincubated with the indicated amounts of solubilized GST-fusion central loop of GLUT4 for 1 hr at 4° C in PLD assay buffer containing 1% Triton X-100 and 5% glycerol. After a brief centrifugation, the precipitated complexes were washed three times with the same buffer before being loaded onto a polyacrylamide gel. FIG. 11 shows a direct binding of purified PLD1 to the cytoplasmic central loop of GLUT4.

### EXAMPLE 8

### Interaction between PLD2 and Heat Shock 70

(1) Identification of p70 as HSP70

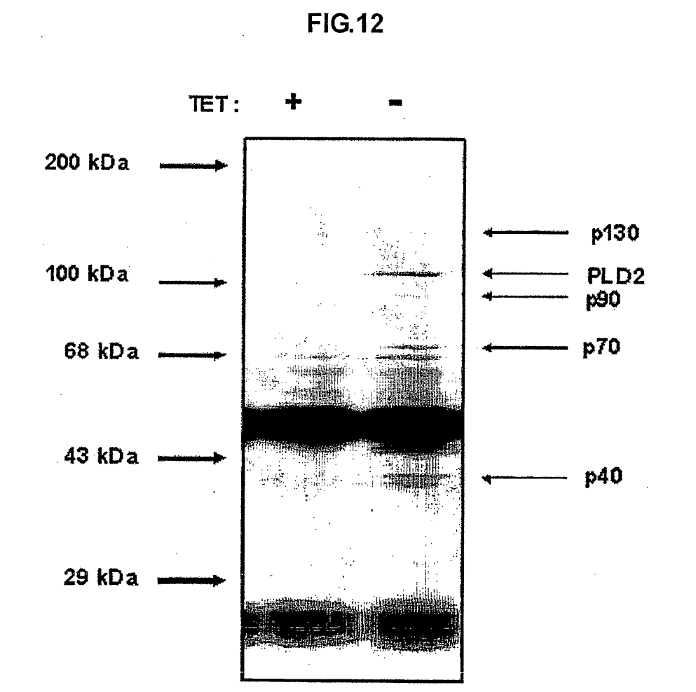

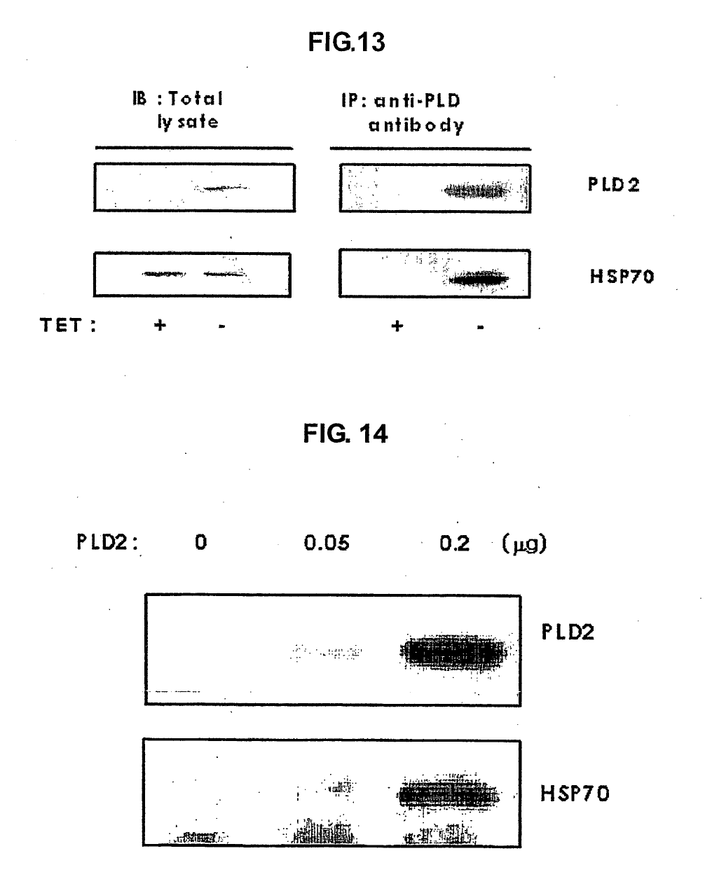

PLD2-inducible PC12 cells were subcultured in the presence or absence of 0.5 g/ml tetracycline for 24 h. Cells were lysed in lysis buffer (50 mM HEPES, pH 7.3, 3 mM EGTA, 2 mM CaCl₂, 3 mM MgCl₂, 80 mM KC1 and 1 mM dithiothreitol, 1% Triton X-100, and 1% sodium cholate) containing protease inhibitors. After centrifugation (12,000×g for 15 min), equal amount of soluble extract was incubated with anti-PLD antibody immobilized on protein A Sepharose. After 4 h of incubation, immune complexes were washed four times with lysis buffer before separated by SDS-PAGE. As shown in FIGS. 12 and 13, HSP70 is detected as a PLD2-binding protein.

(2) Direct Interaction of HSP70 and PLD2

Protein peptide fingerprinting analysis was performed as described (17). In brief, the candidate band was excised from the gel and digested with trypsin. A 1 μl aliquot of the total digest (total volume 30 μl) was used for peptide mass fingerprinting. The masses of the tryptic peptides were measured with a Bruker Reflex III mass spectrometer. Matrix-assisted laser desorption/ionization (MALDI) was performed with α-cyano-4-hydroxycinnamic acid as the matrix. Trypsin autolysis products were used for internal calibration. Delayed ion extraction resulted in peptide masses with better than 50 ppm mass accuracy on average. Comparison of the mass values against the Swiss-Protein database was performed by Profound. FIG. 14 shows a direct interaction of HSP70 and PLD2.

### EXAMPLE 9

### Interaction between PLD2 and Dynamin

(1) Identification of Dynamin as PLD-Interacting Protein from Rat Brain

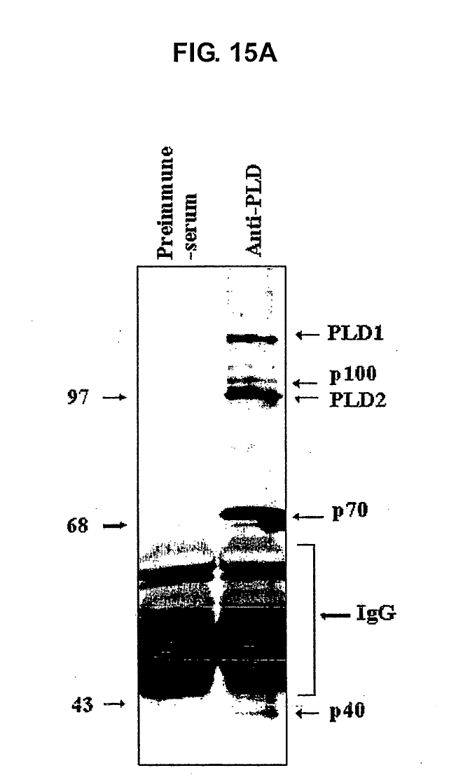

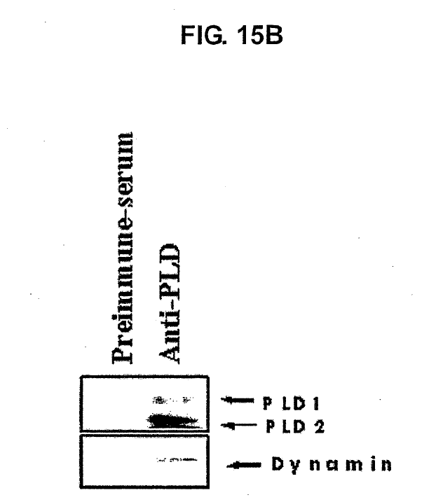

Rat brains were lysed with buffer A (20 mM Tris/HCl, pH 8.0, 150 mM NaCl, 1 mM MgCl₂, 1 mM EGTA, 1% Triton X-100, 1% cholic acid) by sonication. The lysates were centrifuged at 100,000×g at 4° C. for 30 min, and the supernatants were incubated with anti-PLD antibody immobilized to Protein A-Sepharose beads. The resulting immune complexes were pelleted down and washed three times with buffer A and subjected to SDS-PAGE followed by silver staining. After stained with silver staining, the candidate band was excised from the gel and digested with trypsin as described. A 1 μl aliquot of the total digest (total volume 30 μl) was used for peptide mass fingerprinting. The masses of the tryptic peptides were measured with a Bruker Reflex III mass spectrometer. Matrix-assisted laser desorption/ionization (MALDI) was performed with α-cyano-4-hydroxycinnamic acid as the matrix. Matrix-assisted laser desorption/ionization (MALDI) was performed with α-cyano-4-hydroxycinnamic acid as the matrix. Trypsin autolysis products were used for internal calibration. Delayed ion extraction resulted in peptide masses with better than 50 ppm mass accuracy on average. Comparison of the mass values against the Swiss-Protein database was performed using Peptide Search. As shown in FIGS. 15A and 15B, dynamin is detected as a PLD2-binding protein.

(2) Dynamin Interacts with PLD2 in a GTP-Dependent Manner

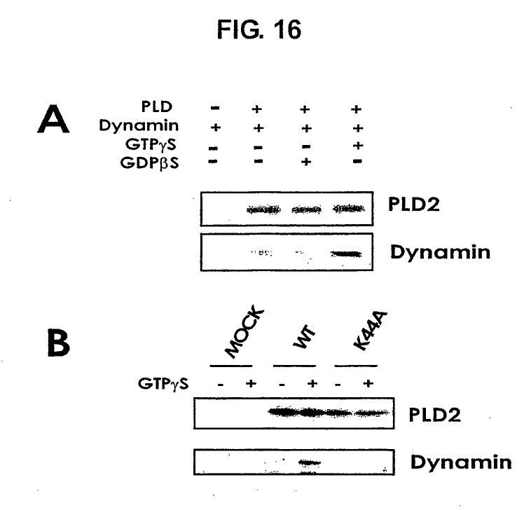

In vitro binding of purified dynamin, DYN-K44A, various GST fused proteins of dynamin PLD2, and PLD2 domains was performed with 300 ml of PH buffer containing 1% Triton X-100, 1% cholic acid at 4° C. for 3 h in the presence of 500 mM GTP□S or GDP□S. After washing three times with the binding buffer, the samples were subjected to SDS-PAGE followed by immunoblot analysis. FIG. 16 shows a direct interaction of dynamin and PLD2 in a GTP-dependent manner.

### EXAMPLE 10

### Interaction between PLD2 and Munc 18

(1) Munc-18-1 was Identified as a PLD-Interacting Protein from Rat Brain Membrane.

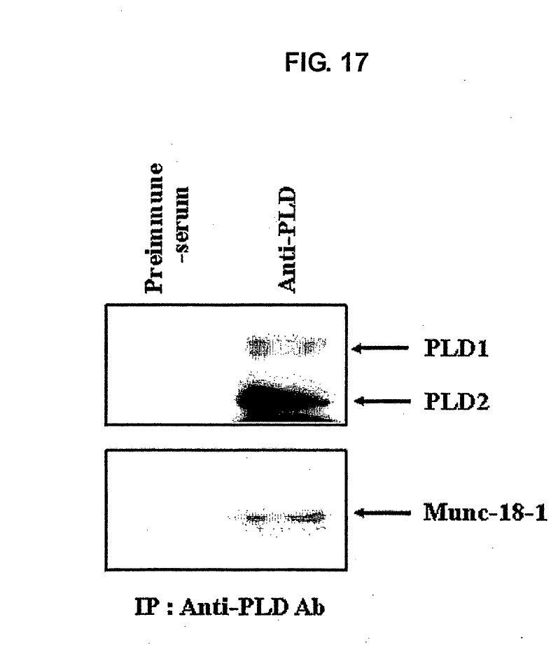

Rat brains (3 g) were lysed with buffer A (20 mM Tris/HCl, pH 8.0, 150 mM NaCl, 1 mM MgCl₂, 1 mM EGTA, 1% Triton X-100, 1% cholic acid) by homogenization. The lysates were centrifuged at 100,000×g at 4° C. for 30 min, and the supernatants were incubated with anti-PLD antibody immobilized to Protein A-Sepharose beads. As shown in FIG. 17, munc-18-1 is identified as a PLD-interacting protein.

(2) Munc-18-1 Direct Interacts with PLD2 in a Dose-Dependent Manner.

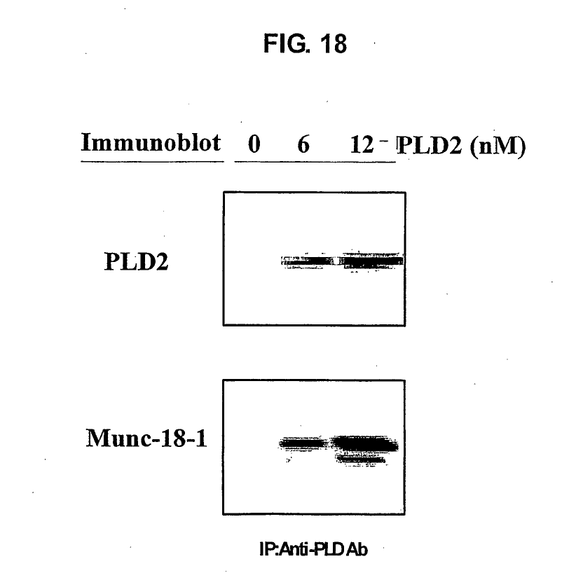

Cells were lysed with PLD assay buffer (50 mM HEPES/NaOH, pH 7.3, 3 mM EGTA, 3 mM CaCl₂, 3 mM MgCl₂, and 80 mM KC1) containing 1% Triton X-100, 1% cholic acid, 1 mM phenylmethylsulfonyl fluoride. After brief sonication, the cell lysates were centrifuged at 100,000×g for 30 min at 4° C. The supernatants (1 mg of protein) were incubated -with anti-PLD antibody-immobilized on protein A resin for 6 h at 4° C. Proteins were denatured by boiling for 5 min at 95° C. in a Laemmli sample buffer, separated by SDS-PAGE, and immunoblot analysis was performed as described previously. In vitro binding was performed in 300 μl of PLD assay buffer (50 mM HEPES/NaOH, pH 7.3, 3 mM EGTA, 3 mM CaCl₂, 3 mM MgCl₂, and 80 mM KC1) containing 0.1% Triton X-100 and 0.1% cholic acid at 4° C. for 2 h. FIG. 18 shows a direct interaction of munc-18-1 and PLD2 in a dose-dependent manner.

### EXAMPLE 11

### Interaction between PLD2 and Tubulin

(1) p55 Protein was Co-Immunoprecipitatd with PLD2 at PLD2 Activity Turn Off Time.

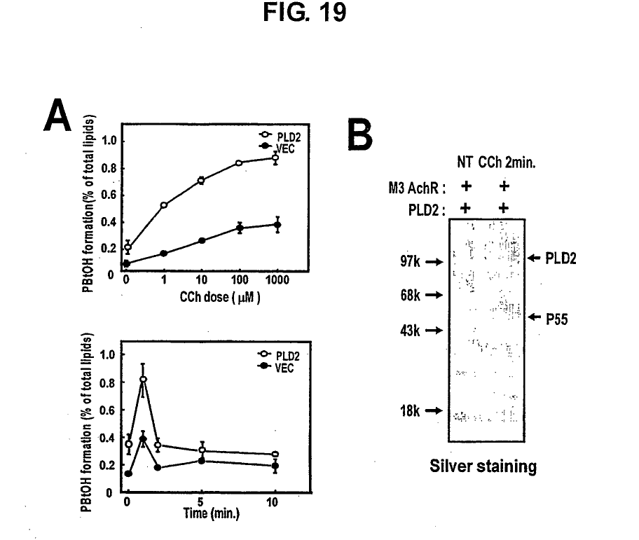

The cultured cells were harvested and lysed with PLD assay buffer (50 mM Hepes/NaOH, pH 7.3, 3 mM EGTA, 3 mM CaCl₂.3 mM MgCl₂, 80 mM KCl) containing 0.5% TX-100, and 1% Cholic acid, 1 mM PMSF, 1 μg/ml leupeptin, and 5 μg/ml aprotinin. After a brief sonication, the lysates were centrifuged at 100,000×g for 1 hr, and the cell extracts were incubated respectively with immobilized anti-PLD antibody for overnight. After a brief centrifugation, the co-immunoprecipitated complexes were washed five times with the same buffer before being loaded onto a gel. In vivo PLD activity was determined as described previously. In brief, vector or human PLD2-transfected COS-7 cells were cultured for 48 h. The cells were loaded with [³H]myristic acid (10 μCi/ml) for 4 h and then washed twice with DMEM. And COS-7 cells were pre-incubated with Nocodazole and Taxol for 20 min. The loaded cells were incubated with 0.4% butanol for 5 min and, scraped into 0.8 ml of methanol and 1 M NaCl (1:1), and mixed with 0.4 ml of chloroform. The organic phases were dried, and the lipids were separated by thin-layer chromatography on silica-gel plates. The PLD activity of PLD2-overexpressing PC12 cells was determined using the same procedures. The amount of [³H]phosphatidylbutanol formed was expressed as a percentage of the total ³H-lipid to account for cell labeling efficiency differences. As shown in FIG. 19, a 55-kDa protein is detected as a PLD2-binding protein.

(2) The 55-kDa Protein Precipitated with PLD2 in COS-7 Cells was Identified as α-, β-tubulin.

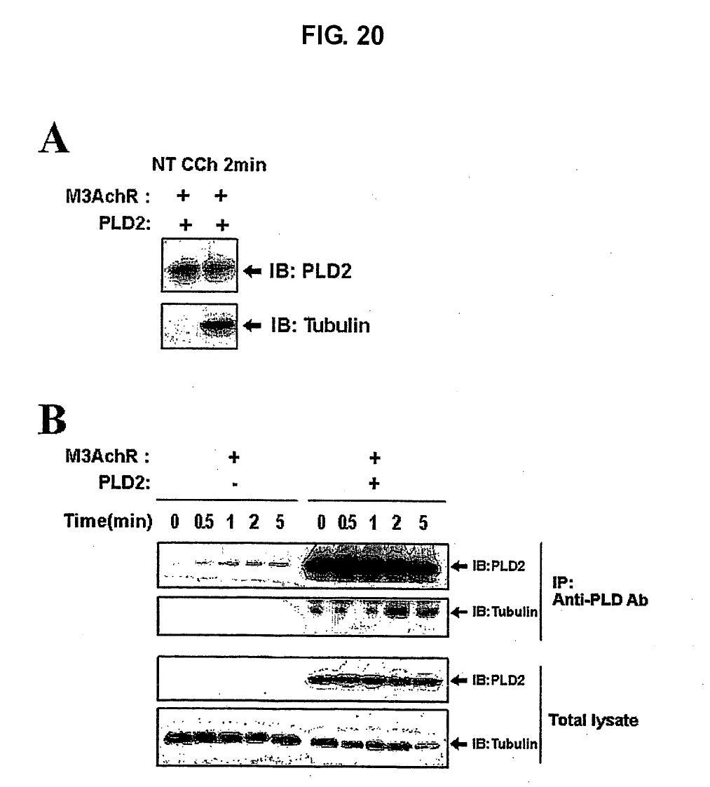

The technique used in this example followed as described previously. In brief, the fraction containing 55-kDa protein (p55) after co-immunoprecipitation from COS-7 cells was separated by SDS-PAGE, and the band corresponding to p55 was excised and digested with trypsin (Roche Molecular Biochemicals) for 6 h at 37° C. Delayed ion extraction resulted in peptide masses with better than 50 ppm mass accuracy on average. Using the amino acid sequences and the mass numbers of the tryptic peptides of p50, the Swiss-Protein database was searched for a protein match. In vitro binding of all the GST fusion PLD2 fragment proteins and immune complex with tubulin was performed in the PLD assay buffer (50 mM Hepes/NaOH, pH 7.3, 3 mM EGTA, 3 mM CaCl₂.3 mM MgCl₂, 80 mM KCl) containing 0.1% Triton X-100, 1 mM PMSF, 1 μg/ml leupeptin, and 5 μg/ml aprotinin for 20 min at 37° C. After a brief centrifugation, the precipitated complexes were washed five times with the same buffer before being loaded onto a polyacrylamide gel. FIG. 20 shows that the 55-kDa protein precipitated with PLD2 in COS-7 cells is α- and β-tubulin.

### EXAMPLE 12

### Interaction between PLD2 and nNOS

(1) Neuronal Nitric Oxide Synthase (nNOS) was Identified as a PLD-Interacting Protein in Rat Brain.

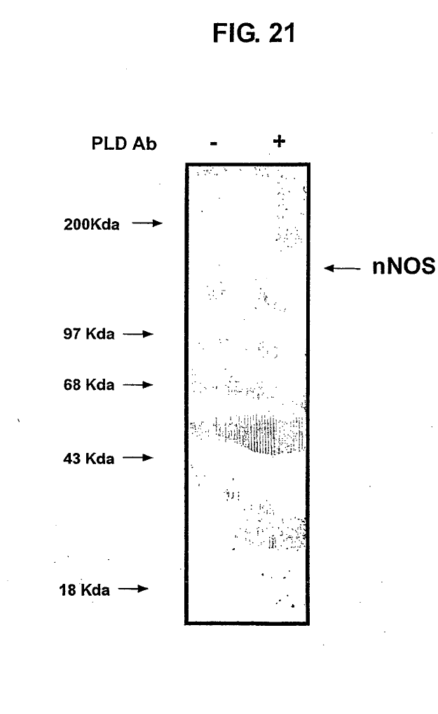

The same procedures as in Example 9 (1) were repeated to identify nNOS as a PLD-interacting protein in rat brain (see FIG. 21).

(2) nNOS More Strongly Interacted with PLD2 than PLD1 in COS-7 Cells

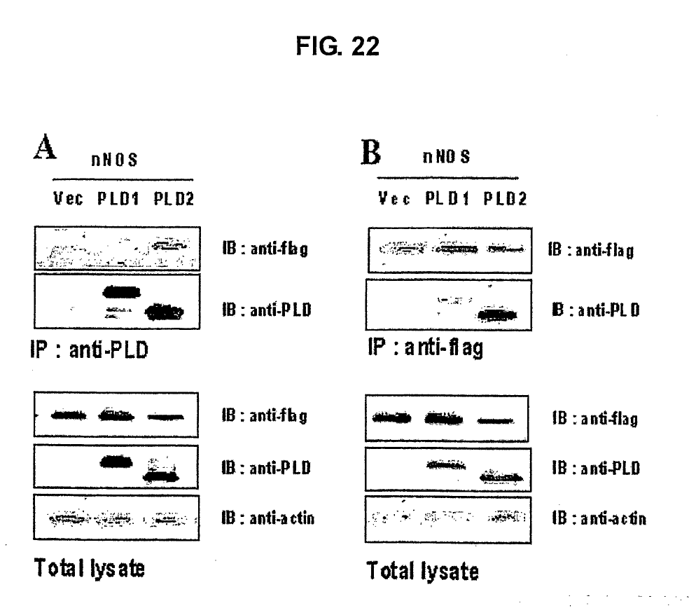

The same procedures as in Example 7 (1) were repeated using pcDNA 3.1 vector harboring PLD1 and pCMV-Flag-2 vector encoding nNOS or pcDNA 3.1 vector harboring PLD2 and pCMV-Flag-2 vector encoding nNOS. FIG. 22 shows a strong interaction between nNOS and PLD2.

### EXAMPLE 13

### Interaction between PLD2 and Integrin Beta 3

(1) PLD Directly Interacts with Integrin Beta 3, 5 Cytosolic Tail.

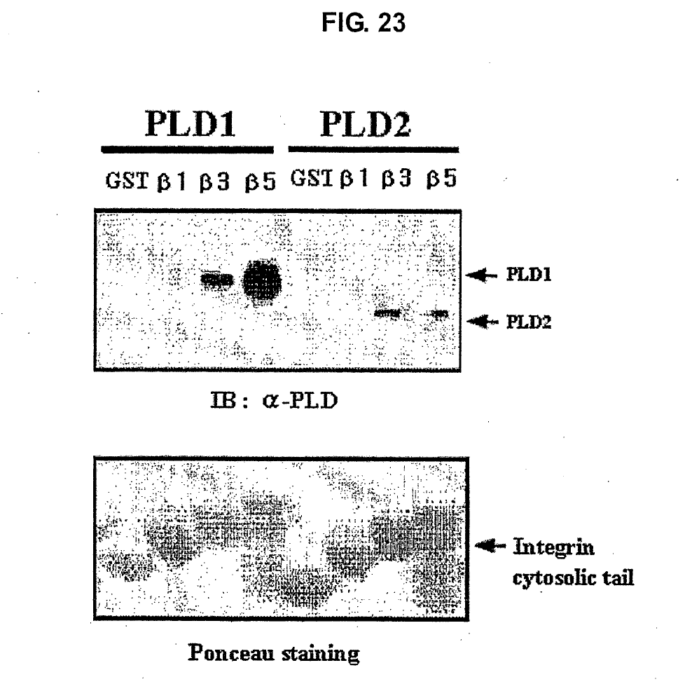

The same procedures as in Example 7 (2) were repeated to identify a direct interaction between PLD2 and integrin beta 3, 5 cytosolic tail (see FIG. 23).

(2) PLD Interacts with Integrin Beta3 in COS 7 Cells.

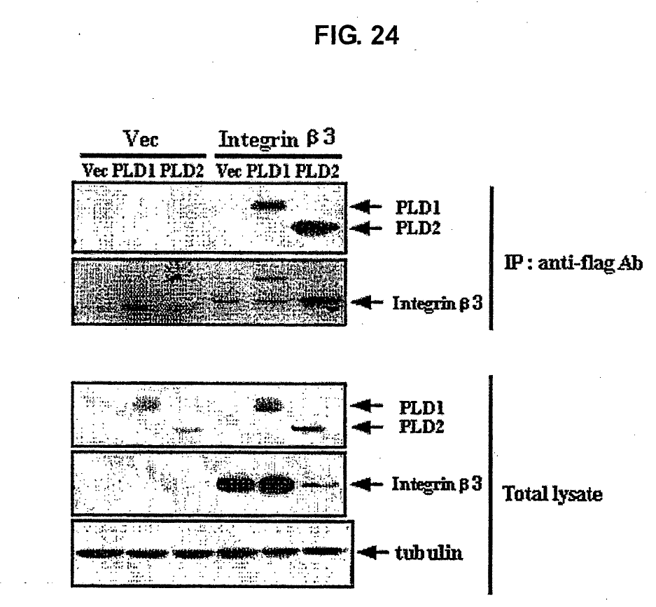

The same procedures as in Example 7 (1) were repeated using pcDNA 3.1 vector harboring PLD1 and pCMV-Flag-2 vector encoding integrin beta3 or pcDNA 3.1 vector harboring PLD2 and pCMV-Flag-2 vector encoding integrin beta3. FIG. 24 shows PLD directly interacts with integrin beta 3 in COS7 cells.

### EXAMPLE 14

### Interaction between PLD1 & 2 and mTOR

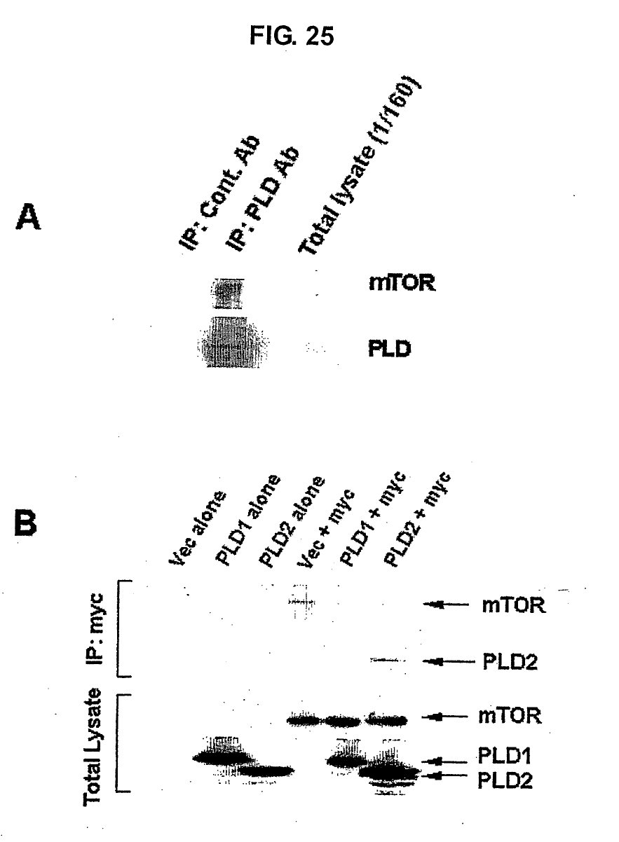

Binding mapping analysis and translation assay were performed with PLD1 & 2, using the procedure as in Example 7 (1). FIG. 25 shows an interaction between PLD 1 & 2 and mTOR.

### EXAMPLE 15

### Interaction between PLD2 and PIP3

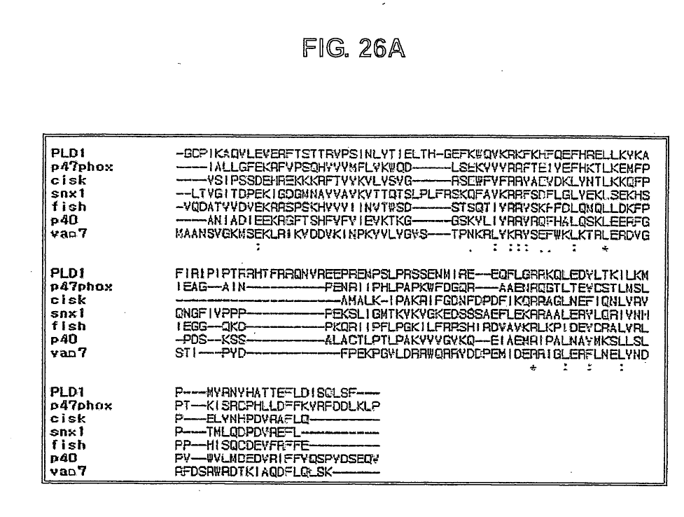

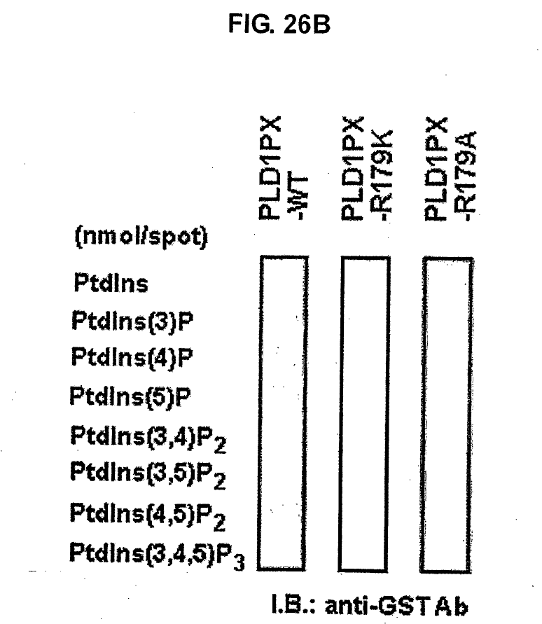

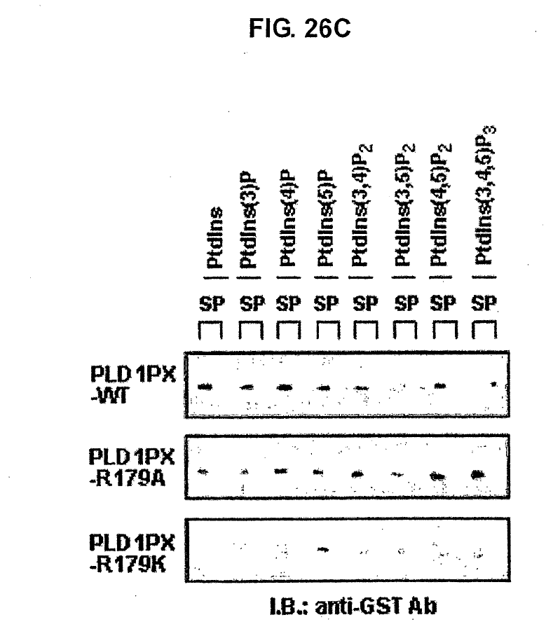

A protein-lipid overlay assay was performed using GST fusion proteins as previously described. Lipids were dissolved in chloroform: methanol: water (1: 2: 0.8 volume ratio) and 1 μl aliquotes containing 0.2, 0.5, and 1 nmol phosphoinositides were spotted onto Hybond-C extra-membranes (Amersham Pharmacia Biotech). After drying at room temperature for 1 h, the membranes were blocked in 3% fat-free BSA (Sigma-Aldrich) in TNE (50 mM Tris-HCl, pH 7.5,150 mM NaCl, 1 mM EDTA) with 0.1% Tween-20 at 4° C. overnight. Membranes were then incubated with 1 μg/ml of purified GST-PLD1 wild type, R179K, or R179A mutant respectively at room temperature for 1 h, washed five times with TNE with 0.1% Tween-20, and incubated with anti-GST antibody. After 1 h washing, the membranes were further incubated with horseradish peroxidase-conjugated goat anti-mouse antibody for 1 h at room temperature and then washed again for 1 h. The signals were detected by enhanced chemiluminescence kit. Phospholipids vesicles composed of 53 μM phosphatidylcholine (PE), 3.3 μM phosphatidylcholine (PC), and 4.6 μM phosphoinositides (Ptdlns) were incubated with 10 ng of purified GST-PLD1 PX domains in 150 μl buffer containing 50 mM Hepes-NaOH, pH 7.5, 3 mM MgCl₂, 2 mM CaCl₂, 3 mM EGTA, and 80 mM KCl as previously described (Kim et al., 1998). After incubation at 37° C. for 15 min, the reaction mixtures were centrifuged at 300,000×g for 30 min in a TL-100 ultracentrifuge (Beckman). The resulting supernatants and pellets were subjected to 8% SDS-PAGE and immunoblotted using anti-GST antibody. FIGS. 26A, 26B, and 26C show shows PLD1 PX domain has high affinity with phosphoinositide-3-phosphate (PIP3).

While this invention has been particularly shown and described with reference to preferred embodiments thereof, it will be understood by those skilled in the art that various changes in form and details may be made therein without departing from the spirit and scope of the invention as defined by the appended claims.

What is claimed is:

1. An isolated peptide complex comprising:
a first peptide selected from the group consisting of:
(a1) phospholipase D (PLD),
(a2) a PLD variant,
(a3) a PLD fragment, and
(a4) a fusion peptide containing (a1), (a2), or (a3); and
a second peptide selected from the group consisting of
(b1) actin, glyceraldehyde-3-phosphate dehydrolase (GAPDH), Akt1, glucose transporter 4 (GLUT4), mammalian target of rapamycin (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), or dopamine transporter (DAT),
(b2) a variant of (b1),
(b3) a fragment of (b1), and
(b4) a fusion peptide containing (b1), (b2), or (b3).

2. The isolated peptide complex of claim 1, wherein the first peptide is PLD and the second peptide is selected from the group consisting of actin, glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT).

3. The isolated peptide complex of claim 1, wherein the first peptide is the fusion peptide containing PLD, a PLD variant or a PLD fragment.

4. The isolated peptide complex of claim 1, wherein the second peptide is the fusion peptide containing one or more peptide selected from the group consisting of actin, glyceraldehyde-3-phosphate dehydrolase (GAPDH), Akt1, glucose transporter 4 (GLUT4), mammalian target of rapamycine (mTOR), heat shock protein 70 (hsp70), dynamin, munc 18, tubulin, n-nitric oxide synthase (nNOS), integrin beta 3, guanine nucleotide exchange factor—H1 (GEF-H1), V-ATPase, phosphoinositide-3-phosphate (PIP3), dopamine transporter (DAT), a variant thereof, and a fragment thereof.

5. The isolated peptide complex of claim 1, wherein the first peptide is linked to the second peptide by a covalent bond.

6. A screening method for modulators of the peptide complex according to claim 1, which comprises:
providing the isolated peptide complex;
contacting the isolated peptide complex with a test compound; and
detecting an interaction between the test compound and the isolated peptide complex and/or an interaction change between the first peptide and the second peptide.

7. A screening method for modulators of an interaction between a first peptide selected from the group consisting of
(a1) phospholipase D (PLD),
(a2) a PLD variant,
(a3) a PLD fragment, and
(a4) a fusion peptide containing (a1), (a2), or (a3); and
a second peptide selected from the group consisting of
(b1) actin, aldolase, collapsin response mediator molecule-2 (CRMP-2), phospholipase C-γ1 (PLC-γ1), glyceraldehyde-3-phosphate dehydrolase (GAPDH), Akt1, glucose transporter 4 (GLUT4), mammalian target of rapamycin (mTOR), heat shock protein 70 (hsp70), dynamin, munc 18, tubulin, n-nitric oxide synthase (nNOS), integrin beta 3, guanine nucleotide exchange factor—H1(GEF-H1), V-ATPase, phosphoinositide-3-phosphate (PIP3), or dopamine transporter (DAT),
(b2) a variant of (b1),
(b3) a fragment of (b1), and
(b4) a fusion peptide containing (b1), (b2), or (b3),
which comprises:
contacting the first peptide with the second peptide in presence of a test compound; and
detecting an interaction between the first peptide and the second peptide.

8. The screening method of claim, wherein at least one of the first and second peptides is a fusion peptide having a detectable tag.

9. The screening method claim, wherein the contacting step is conducted in a substantially cell free environment.

10. The screening method claim, wherein the interaction or interaction change between the first peptide and the second peptide is determined in a host cell.

11. The screening method claim, wherein the detecting comprises measuring the amount of the peptide complex formed with the first and second peptides.

12. The screening method claim, further comprising generating a data set defining one or more selected test compounds.

13. The screening method claim 12, wherein the data set is in a transmittable form.


SEQUENCE LISTING (15 sequences)

SEQ ID NO: 1 - protein, 6 aa, Rattus norvegicus
LDLAGR

SEQ ID NO: 2 - protein, 7 aa, Rattus norvegicus
ILAPPER

SEQ ID NO: 3 - protein, 10 aa, Rattus norvegicus
GYSFTTTAER

SEQ ID NO: 4 - protein, 11 aa, Rattus norvegicus
HQGVMVGMGQK

SEQ ID NO: 5 - protein, 13 aa, Rattus norvegicus
QEYDESGPSIVHR

SEQ ID NO: 6 - protein, 16 aa, Rattus norvegicus
SYELPDGQVITIGNER

SEQ ID NO: 7 - protein, 18 aa, Rattus norvegicus
VAPEEHPVLLTEAPLNPK

SEQ ID NO: 8 - protein, 356 aa, Rattus norvegicus
PHSYPALSAEQKKELSDIALRIVAPGKGILAADESVGSMAKRLSQIGVENTEENRRLYRQVLFSADDRVKKCIGGVIFFHETLYQKDDNGVPFVRTIQEKGILVGIKVDKGVVPLAGTDGETTTQGLDGLLERCAQYKKDGADFAKWRCVLKISDRTPSALAILENANVLARYASICQQNGIVPIVEPEILPDGDHDLKRQFVEKVLAAVYKALSDHHVYLEGTLLKPNMVTPGHACPIKYSPEEIAMTALRRTVPPAVPGVTFLSGGQSEEEASLNLNAINRCSLPRPWALTFSYGRALQASALSAWRGQRDNAGAATEEFIKRAEMNGLAAQGKYEGSGDGGAAAQSLYVANAY

SEQ ID NO: 9 - protein, 136 aa, Homo sapiens
GCPIKAQVLEVERFTSTTRVPSINLYTIELTHGEFKWQVKRKFKHFQEFHRELLKYKAFIRIPIPTRRHTFRRQNVREEPREMPSLPRSSENMIREEQFLGRRKQLEDYLTKILKMPMYRNYHATTEFLDISQLSF

SEQ ID NO: 10 - protein, 120 aa, Homo sapiens
IALLGFEKRFVPSQHYVYMFLVKWQDLSEKVVYRRFTEIYEFHKTLKEMFPIEAGAINPENRIIPHLPAPKWFDGQRAAENRQGTLTEYCSTLMSLPTKISRCPHLLDFFKVRPDDLKLP

SEQ ID NO: 11 - protein, 103 aa, Homo sapiens
VSIPSSDEHREKKKRFTVYKVLVSVGRSEWFVFRRYAEVDKLYNTLKKQFPAMALKIPAKRIFGDNFDPDFIKQRRAGLNEFIQNLVRYPELYNHPDVRAFLQ

SEQ ID NO: 12 - protein, 121 aa, Homo sapiens
LTVGITDPEKIGDGMNAYVAYKVTTQTSLPLFRSKQFAVKRRFSDFLGLYEKLSEKHSQNGFIVPPPPEKSLIGMTKVKVGKEDSSSAEFLEKRRAALERYLQRIVNHPTMLQDPDVREFL

SEQ ID NO: 13 - protein, 117 aa, Mus musculus
VQDATVVDVEKRRSPSKHYVYIINVTWSDSTSQTIYRRYSKFFDLQMQLLDKFPIEGGQKDPKQRIIPFLPGKILFRRSHIRDVAVKRLKPIDEYCRALVRLPPHISQCDEVFRFFE

SEQ ID NO: 14 - protein, 120 aa, Homo sapiens
ANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDSEQV

SEQ ID NO: 15 - protein, 122 aa, Saccharomyces cerevisiae
MAANSVGKMSEKLRIKVDDVKINPKYVLYGVSTPNKRLYKRYSEFWKLKTRLERDVGSTIPYDFPEKPGVLDRRWQRRYDDPEMIDERRIGLERFLNELYNDRFDSRWRDTKIAQDFLQLSK